(19) United States
(12) Patent Application Publication
(10) Pub. No.: US 2025/0276021 A1
(43) Pub. Date: Sep. 4, 2025

(54) ISOLATION, ENRICHMENT, AND EXPANSION OF HUMAN ROD PROGENITOR CELLS AND EXTRACELLULAR VESICLES DERIVED THEREFROM

(71) Applicants: The Schepens Eye Research Institute, Inc., Boston, MA (US); RESEARCH FOUNDATION OF THE CITY UNIVERSITY OF NEW YORK, NEW YORK, NY (US)

(72) Inventors: Michael J. Young, Boston, MA (US); Pierre Colombe Dromel, Boston, MA (US); Deepti Singh, Boston, MA (US); Stephen Redenti, Sea Cliff, NY (US)

(21) Appl. No.: 18/858,911

(22) PCT Filed: May 5, 2023
(86) PCT No.: PCT/US2023/021227

§ 371 Date: Oct. 22, 2024

Related U.S. Application Data
(60) Provisional application No. 63/339,252, filed on May 6, 2022.

(51) Int. Cl. A61K 35/30; A61K 47/10; A61K 47/34; A61P 27/02; C12N 5/00; C12N 5/0793

(52) U.S. Cl. CPC A61K 35/30; A61K 47/10; A61K 47/34; A61P 27/02; C12N 5/0018; C12N 5/062; C12N 2500/02; C12N 2501/11; C12N 2501/119

(57) ABSTRACT

Described herein are methods for producing enriched populations of progenitor rod photoreceptor cells (PRPs) and EVs derived therefrom, as well as methods of using the PRPs and EVs to diagnose and treat retinal disease.

### CLAIM OF PRIORITY

This application claims the benefit of U.S. Provisional Patent Application Ser. No. 63/339,252, filed on May 6, 2022. The entire contents of the foregoing are hereby incorporated by reference.

### TECHNICAL FIELD

Described herein are methods for producing enriched populations of progenitor rod photoreceptor cells (PRPs) and extracellular vesicles (EVs) derived therefrom, as well as methods of using the PRPs and EVs to diagnose and treat retinal disease.

### BACKGROUND

Retinal degenerative diseases, which lead to the death of rod and cone photoreceptor cells, are the leading cause of inherited vision loss worldwide. Retinitis Pigmentosa, for example, is a disease in which rods photoreceptor die first leading to the death of cone photoreceptors. Induced pluripotent or embryonic stem cells (iPSCs/ESCs) have been proposed as a possible source of new photoreceptors to restore vision in these conditions. Studies carried out in mouse models of retinal degeneration over the past decade have highlighted several limitations for cell replacement or neuroprotection in the retina, such as poor integration of grafted cells in the host retina and high dosage needed to neuro-protect the retina.

### SUMMARY

Described herein are methods of providing a population of progenitor rod photoreceptor cells (PRPs). The methods can include providing an initial population comprising retinal cells from a mammal, preferably a fetal mammal (alternatively, the initial population can comprise cells from a retinal or optic cup organoid generated from iPSC or ESC). Cells expressing CD73 and CD276 (and optionally Cd11b) are isolated from the initial population to provide a substantially purified population of PRPs.

In some embodiments, isolating cells expressing CD73 and CD276 (and optionally Cd11b) comprises using fluorescence activated cell sorting (FACS) or Magnetic-activated cell sorting (MACS), optionally in a microfluidic device. For example, the methods can include contacting the initial population of cells with antibodies that bind to CD73 and CD276, and isolating cells to which antibodies to both are bound.

In some embodiments, the methods further comprise culturing the initial population comprising retinal cells before isolating cells expressing CD73 and CD276 (and optionally Cd11b).

The methods can include optionally culturing the substantially purified population of CD73+/CD276+ PRPs obtained. In some embodiments, the substantially purified population of PRPs is cultured in media comprising serum or serum replacement, EGF, ad FGF. In some embodiments, the substantially purified population of PRPs is cultured in hypoxic conditions (e.g., 3-10% O₂). In some embodiments the hPRPs are also CD11b+.

In some embodiments, the initial population comprising retinal cells is from a fetal mammal, e.g., a human.

Also provided herein are substantially purified populations of PRPs produced by a method described herein. In some embodiments, at least 75%, e.g., at least 80%, 85%, 90%, or 95%, of the population comprises cells expressing both CD73 and CD276 (and optionally Cd11b). Further provided are the substantially purified populations of PRPs for use in treating a subject who has a condition associated with loss of retinal rod or cone photoreceptors, as described herein. In some embodiments, the condition associated with loss of retinal rod or cone photoreceptors is an inherited retinal degenerative disease (IRD), optionally cone-rod dystrophy, retinitis pigmentosa (e.g., LCA), or Stargardt's disease; or macular degeneration, e.g., dry macular degeneration. Also provided herein are compositions comprising PRPs as described herein (e.g., that are at least 75%, e.g., at least 80%, 85% 90%, or 95%, of the population comprises cells expressing both CD73 and CD276 (and optionally Cd11b)), optionally formulated in a physiologically acceptable buffer or polymeric gel, e.g., as described herein, optionally gelatin-hyaluronic acid (HA) hydrogel, e.g., a composition comprising gelatin hydroxyphenylpropionic acid (gelatin-HPA) and hyaluronic acid-tyramine (HA-Tyr).

Additionally, provided herein are methods for treating a subject who has a condition associated with loss of retinal rod photoreceptors. The methods can comprise administering to the subject (e.g., by injection) a therapeutically effective amount of PRPs obtained by a method described herein, preferably wherein at least 75%, e.g., at least 80%, 85%, 90%, or 95%, of the population comprises cells expressing both CD73 and CD276 (and optionally Cd11b), optionally formulated in a physiologically acceptable buffer or polymeric gel, e.g., as described herein, optionally gelatin-hyaluronic acid (HA) hydrogel, e.g., a composition comprising gelatin hydroxyphenylpropionic acid (gelatin-HPA) and hyaluronic acid-tyramine (HA-Tyr)).

Further, provided herein are methods of obtaining or providing extracellular vesicles (EVs) from rod photoreceptor progenitor cells. The methods can comprise providing a substantially purified population of PRPs as described herein; maintaining the substantially purified population of PRPs in culture in media, preferably wherein the media is in contact with the PRPs in the culture for at least 12, 18, 24, 36, 48, or 72 hours; removing the media from the culture; and isolating EVs from the media.

In some embodiments, isolating EVs comprises ultracentrifuging the media to obtain a pellet comprising EVs. An exemplary ultracentrifuge protocol comprises 3 steps: 1500 g for 30 min; 10,000 g for about 20 min; and 15,000 g for 30 min. In some embodiments, the methods further comprise lyophilizing the isolated EVs.

Also provided herein are compositions comprising EVs obtained by a method described herein. In some embodiments, the EVs are in a polymeric gel scaffold, e.g., a polymeric scaffold comprising polycaprolactone (PCL), polylactic-glycolic acid (PLGA), polyethylene gly % col (PEG), polylactic acid (PLA), polyetherimide (PEI), PNIPAAM, or hydroxy ethyl-methacrylate. Also provided are the compositions for use in treating a subject who has a condition associated with loss of retinal rod or cone photoreceptors. Additionally provided herein are methods for treating a subject who has a condition associated with loss of retinal rod or cone photoreceptors, as described herein. The methods can comprise administering to the subject a therapeutically effective amount of PRPs obtained by a method described herein.

In some embodiments, the condition associated with loss of retinal rod or cone photoreceptors is an inherited retinal degenerative disease (IRD), optionally cone-rod dystrophy, retinitis pigmentosa (e.g., LCA), or Stargardt's disease; or macular degeneration, e.g., dry macular degeneration.

As used herein, unless otherwise specified the term “about” mean±10% of the indicated number.

Unless otherwise defined, all technical and scientific terms used herein have the same meaning as commonly understood by one of ordinary skill in the art to which this invention belongs. Methods and materials are described herein for use in the present invention: other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1. Summary of exemplary isolation methods for human progenitor rod photoreceptors (hPRP). Cells are isolated from human fetal retina with CD73+/CD276+ markers. Post-sorting cells show high purity >95% for both markers along with other specific rod expressing markers. High viability can be seen both post-sorting and after weeks of cell culture with media as described herein. The markers shown demonstrate maturation and cell lineage.

FIG. 2. Diagram of exemplary neuroprotective human EVs extracted from hPRP Illustrative exemplary diagram showing multiple encapsulated molecules and proteins in EVs along with transmembrane proteins enabling EVs signaling and genesis.

FIG. 3. Illustrative schematic of exemplary EVs manufacturing process. Manufacturing process of extraction of EVs from hPRP along with encapsulation and in vivo injection for potential neuroprotective effect on retinal diseases.

FIG. 4. Total number of cells throughout the process of sorting. Total number of cells in function of time (weeks of culture). Number of cells was measured in T75 flasks using Trypan blue and hemacytometer. The average number of cells after digestion of retina is 2 million and is cultured until reaching 10 million cells. Upon reaching this number cells are sorted; therefore, total number drops down. Then the isolated rod progenitor cells are cultured until reaching high number (40 million) of cells at 5 weeks.

FIG. 5. Brightfield images of rods hPRP post-sorting in T75 flasks. Cells were imaged with Leica Brightfield upright microscope showing successful cell attachment, viability, morphology, and proliferation over 20 days of culture.

FIG. 6. Live sorting and gating with a MACSQUANT cell sorter using a TYTO cartridge (Miltenyi), Clive CD73 and CD276 gating of fetal retinal cells to isolate pure hPRP. A clear population was seen for both CD73 and CD276 and can be isolated with high viability and purity post-sorting.

FIG. 7. TYTO Sorting data analyzed for input, output and negative fractions of cells. Expression of sorting markers (CD73 and CD276) during the sorting process for the input, output and negative fraction of cells. Data was analyzed using the TYTO sorter and the MACSQUANT flow cytometer. It shows the expression of CD73/CD276 positive cells before and after the first sort. CD73/CD276 population starts around 30-40% in the unsorted sample and rises to 95% after sort. Data is shown as mean±SD for 10 replicates (10 different sorts). Statistical one-way ANOVA was performed and shows significant difference between input and output (*p<0.001).

FIG. 8. Live timing and analysis of sorted cells with TYTO. Cell sorter analysis showing triggered event, sort rate and percent of positively sored cells across a 1 h sorting. A constant >90% sorted can be observed for the entire sorting process.

FIG. 9. Raw sorting markers expression analyzed with live flow cytometry. Expression of sorting markers (CD73, CD276) with their specific fluorophore (APC, PE, VioBlue) before and after sort. Data was analyzed suing the MACSQUANT flow cytometer. It shows data of the sort—CD73/CD276 used in two fluorophores. Top right panel line: scatter and histogram plot of CD73/CD276 expression for input. Middle right panel line: scatter and histogram plot of CD73/CD276 expression for output. Top left panel: gating strategy and population number corresponding to the gates. Histogram in both figures shows that, post sorting, CD73 and CD276 positive cell population was increase. This is proven by the higher intensity of the peaks of these markers. A minimum of 50,000 cells was used for each sample in flow cytometry.

FIG. 10. Flow cytometry data evolution for 16-weeks tissue during the process of sorting. Left side shows the gating strategy for cells, singles and DAPI positive events to analyze hPRP. Right side shows the expression of purity markers (Recoverin, CD73, CD276) for both unsorted and hPRP Expression of those 3 rods markers was found to be extremely high and pure in hPRP while being between 20-40% present in the unsorted population. This suggest that our sorting strategy enables to capture a pure population of rod progenitor cells. A minimum of 50,000 cells was used for each sample in flow cytometry Data is shown as mean±SD for 5 replicates (5 different flow experiments).

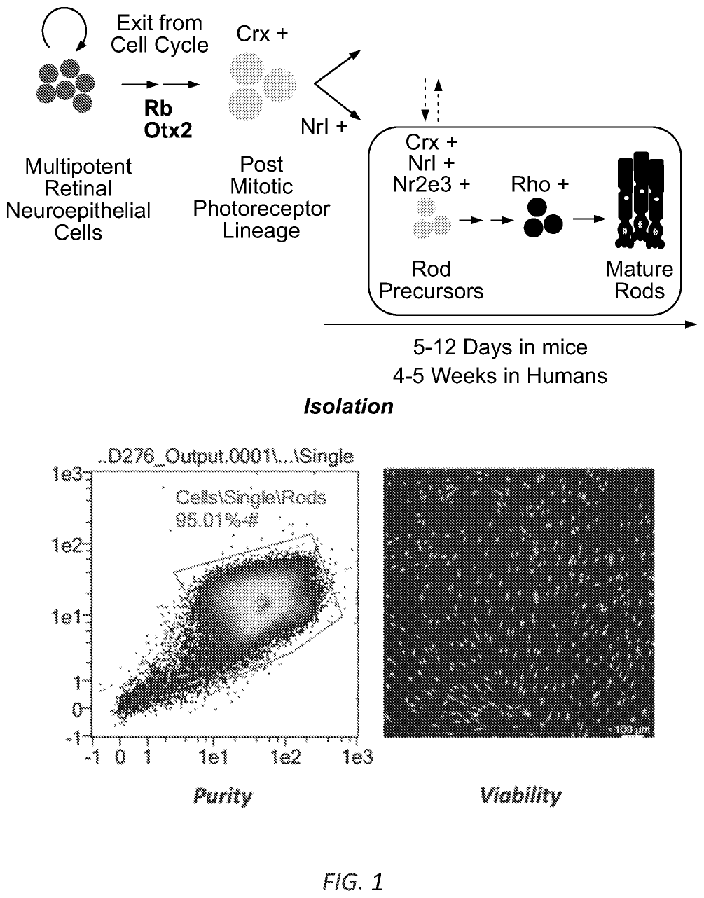

FIG. 1. Flow cytometry data for hPRP vs fetal retina. It shows the differences in all these markers between a sorted hPRP and unsorted fetal retina tissue (also known as hRPC). Overall, rod markers (CD73, CD276, Recoverin, Cone rod homeobox (CRX)) were found to be extremely high in hPRP compared to unsorted retina. Other markers of retinal cells (cones, RGC, bipolar, horizontal and amacrine) were found negative in hPRP. Finally, 15-20% of hPRP were found to be proliferative, with a 50% PAX6 retinal marker expression. A minimum of 50,000 cells was used for each sample in flow cytometry.

FIG. 12. Immuno-staining analysis performed on hPRP with sorting markers. CD73, CD276 and CRX are distinct rod photoreceptors markers and hPRP were stained with same primary antibodies. High expression of these 3 markers can be observed in the IHC images. The nuclear stain used to identify the cells was DAPI.

FIG. 13. Immune staining for different markers for hPRP. Cells were stained with common rod markers (Rhodopsin, Recoverin) along with retinal marker (PAX6) and proliferation marker (K167). Overall, all markers were found in hPRP. Other retinal markers like Calbindin (Amacrine cells), Recoverin (photoreceptors), PKCa (Bipolar) and Rhodopsin (rod photoreceptors) were tested, and expression varied from low to no expression. Nuclei were stained with DAPI and all images are taken at 20× magnification.

FIG. 14. Whole mounted retina of RD1 mouse post-transplantation. In normal cases, RD1 mice at P31 should not possess any cone or any ONL staining due to the fast degeneration process of the modified specie; this is what was observed in control samples. However, in animals which received an injection of hPRP, staining showed here was found. Cone Arresting positive staining was found which suggests that the injection of hPRP in the vitreous has a strong neuroprotective impact on the retina of RD1 mice.

FIG. 15. ONL presence in RD1 mice post-transplantation. In normal cases, RD1 mice at P31 have extremely degenerating retina that can be seen as thinning of ONL or absences of one. To measure the impact of hPRP injection in this fast degenerative mouse mode, we analyzed in a binary fashion the presence of one line of ONL nuclei and gave scores to all animals with multiple sections. As seen in the graph on the left, the score grade was found to be higher for animals which received hPRP injection in the subretinal space and even higher for the vitreous injection. This suggests that the injection of hPRP in the vitreous has a strong neuroprotective impact on the retina of RD1 mice.

FIG. 16. Intraocular pressure (IOP) testing of RD1 mice. In normal cases, RD1 mice at P31 should possess and IOP between 10-20 mmHg, which was observed in the control eye. IOP was found to be increased by the injection of cells in the vitreous while no change was observed for control and subretinal injection. However, IOP in eye with vitreous injection was found to go back to the baseline as soon as 1-week post-transplantation. This study suggests a high safety for the injection of hPRP in the eye of rodents.

FIG. 17. Comparison of Extracellular Vesicle Proteomic Cargo between our EVs and Developing Retina.

FIG. 18. Summary of Vesicle Proteomic Cargo Composition.

FIG. 19. Fold change of total proteome cargo in our EVs, segregated into the 6 categories and subcategories.

FIG. 20. Fold change in protein expression comparison between our EVs and the fetal retina (using only a 5× limit).

FIG. 21. Proteins exclusive to our EVs and proteins absent from the fetal retina.

FIGS. 22A-B. Functional behavior testing in Rho−/− mice by optomotor testing. Mice were injected with either (cell+gel) or SHAM (pure saline water) at 3 weeks and tested weekly for functional retinal behavior with OMR apparatus. A. Visual acuity (VA) of mice measured as threshold of spatial frequency. B. Contrast sensitivity threshold of mice, measured at 0.2 cycle/degree VA. (**p=0005).

FIGS. 23A-C. Cone retinal function testing in Rho−/− mice by ERG. Mice were injected with either (cell+gel) or SHAM (pure saline water) at 3 weeks and tested weekly for cone retinal function with ERG apparatus. A. Photopic B-wave measured at 600 cd.s/m2 for both groups. B. Percent increase calculation for Cell+Gel vs SHAM injections. C. Representative photopic B-wave traces at 6 and 7 weeks. (**p=0.005).

FIGS. 24A-B. Cone retinal function testing in P23H mice by ERG. Mice were injected with either (cell+gel), cells, or SHAM (pure saline water) at 4 weeks and tested weekly for cone retinal function with ERG apparatus, A. Photopic B-wave measured at 600 cd.s/m2 for all groups, B. Percent increase calculation for Cell vs SHAM and Cell+Gel vs SHAM injections. (**p=0.0.00).

### DETAILED DESCRIPTION

Described herein are methods and compositions that address limitations and drawbacks associated with previous approaches in the isolation, purification, and enrichment of rod progenitor cells. Provided are methods for producing enriched populations of human progenitor rod photoreceptor cells (hPRP) and EVs derived therefrom, as well as methods of using the hPRP and EVs to diagnose and treat retinal disease.

Human Progenitor Rot Photoreceptor Cells (hPRP)

Provided herein are purified or enriched populations of rod photoreceptor precursor cells, for example, human progenitor rod photoreceptor cells (hPRP), methods of producing these cells and use of the cells for the treatment of ocular disorders, e.g., retinal degenerative diseases, and other diseases. Described are methods for the isolation, purification and expansion of rod photoreceptor progenitor cells using a microfluidic based cell sorting approach, including a scalable GMP capable protocol for production, e.g., of human progenitor rod photoreceptors, providing the ability to create universal, allogenic, rod photoreceptor cells for preserving and restoring vision, e.g., as seen in FIG. 1.

As shown herein, the combination of CD73/CD276 is useful as a surface marker for labeling and isolating rod cells. CD276 was found to be upregulated in rod progenitor cells and can be released from the membrane and on the cell surface. We used it for labeling the rod progenitor cells (CD276 is exclusively expressed only by rod progenitor cells). CD73 is also known as 5′-nucleotidase ecto (NT5E); exemplary sequences for CD73 are provided in GenBank at Ace. Nos. NP_002517.1 (5′-nucleotidase isoform 1 preproprotein) and NP_001191742.1 (5′-nucleotidase isoform preproprotein). Exemplary sequences for CD276 are provided in GenBank at Acc. Nos. NP_001019907.1 (CD276 antigen isoform a precursor); NP_001316557.1 and NP_079516.1 (CD276 antigen isoform b precursor); and NP_001316558.1 (CD276 antigen isoform c).

Fetal retina cells (e.g., at 10-16 weeks in human embryonic age (fetal age) or an analogous age in development for other mammals such as mice), optionally cultured in hypoxic conditions (3-10% O₂), optionally with a nutrient mix containing serum (such as fetal bovine serum (FBS)) or a serum replacement (such as PHYSIOLOGIX Xeno-Free Serum Replacement (Nucleus Biologics) or KNOCKOUT Serum Replacement (Thermo Fisher)), EGF, and FGF as described herein, provide a timeframe during retinal development where the expression of rod progenitor cells is highest; differentiated iPSC and ESC can also be used. When iPSC/ESC are used, the ESCs/iPSCs are grown into optic cup or retinal organoids and (e.g., at days 40 to 60) the organoids are used as a starting point for sorting using a method described herein, e.g., based on the presence of CD73 and CD276. See, e.g., Nakano et al., Cell Stem Cell. 2012 Jun. 14; 10(6):771-785; Kuwahara et al., Methods Mol Biol. 2017; 1597:17-29: Eiraku et al., Nature. 2011 Apr. 7; 472(7341):51-6; Reh and Fischer, Methods Enzymol. 2006; 419:52-73; Fathi et al., Front Neurosci. 2021 Apr. 20; 15:668857; and Achberger et al., Adv Drug Deliv Rev. 2019 Feb. 1; 140:33-50.

The present techniques can be used for isolation of rod progenitor cells, optionally using microfluidic devices such as the MACSQUANT TYTO sterile cartridge microfluidic cell sorting system (Miltenyi) to provide an enriched population of cells with higher purity and viability than previously reported. The microfluidic device allows capture of cells labeled with both positive markers (CD73/CD276) and/or deletion of the population of cells that was unwanted (all the rest of the cells that are CD73− and/or CD276−). The present methods can be used to obtain populations of cells that are at least 75%, e.g., at least 80%, 0.5%, 90%, or 95%, cells that are positive for (express) both CD73 and CD276. In the present experiments, confirmatory markers Recoverin, CRX, CD73, and CD276 expression showed about 95% of positive for these markers. Furthermore, maintaining the isolated cells using culture conditions described herein allowed for cell proliferation, to obtain large number of hPRP. When transplanted into mouse eyes, the hPRP cells showed an extremely high capacity to provide neuroprotection in a degenerative model. To the best of the present inventors' knowledge, this is the first description of methods to isolate and culture a highly pure rod progenitor cells capable of offering high neuroprotection to the retina.

The hPRP cells can be differentiated to provide a population of human rod photoreceptor cells. A number of protocols are known; an exemplary protocol comprises culturing the cells in DMEM/F12 3:1+KSR/FBS with 0.1% 2-ME (b-mercaptoethanol), 0.2% IGF-1 (IGF1 Recombinant Human Protein), 2% B27 supplement, 1% taurine, and 1 μM 9-cis retinal. The media is changed about every 2 days and the cells mature to rod photoreceptors in about 60 to 90 days.

Extracellular Vesicles (EVs) Derived from Human Progenitor Rod Photoreceptor Cells (hRP).

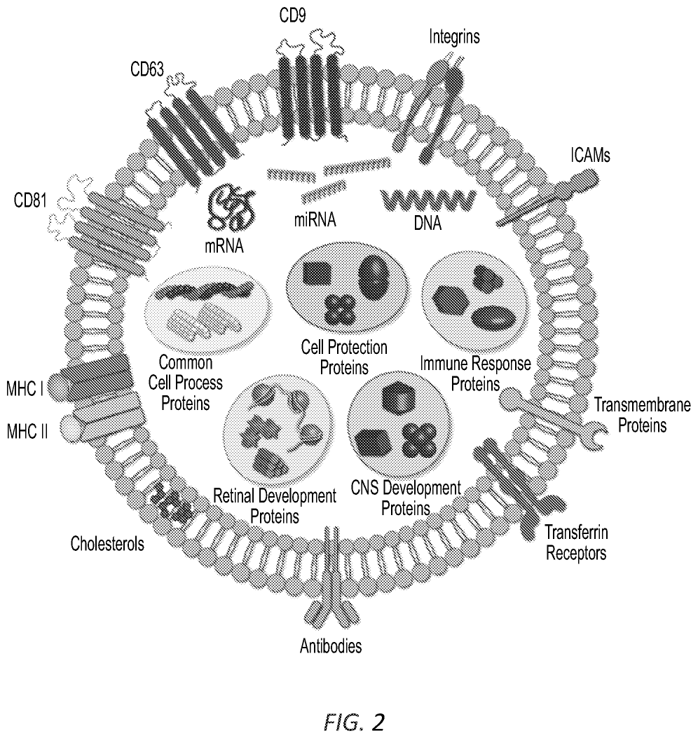

EVs and microvesicles are lipid enclosed cell fragments with diameters ranging from 50 un to 2 mm; they can be derived from almost any cell type, including embryonic stem cells, hematopoietic stems, neurons, and malignant cells. EVs typically have a diameter of 30-130 nm and are formed through the endosomal-sorting complex required for transport (ESCRT). Microvesicles are heterogeneous in size with diameters from 100-2000 nm. While EVs fall within the size range of microvesicles, microvesicles are distinct in formation, secretion, and content. The formation of microvesicles involves interactions between cytoskeletal and phospholipid proteins of the plasma membrane. EVs and microvesicles have been shown to be involved in cell-cell communication via transfer of proteins, mRNA, miRNA and DNA, e.g., as seen in FIG. 2. EVs and microvesicles encapsulate factors representative of cell of origin genotype and phenotype.

Provided herein are EVs derived from human progenitor rod photoreceptor cells (hPRPs) isolated using a method described herein. Some or all of the proteins shown in Table 1 are preferably present in EVs for use in the present methods and compositions, optionally including at least one, two, three four, five, or all seven of Thioredoxin (TXN, also known as RDCVF; NP_001231867.1); EGF containing fibulin extracellular matrix protein 1 (EFEMP1; NP_001034437.1); glutathione S-transferase Mu 1 isoform 1 (GSTM1; NP_000552.2 or NP_666533.1); sarcoglycan delta (SGCD; NP_000328.2, NP_758447.1, or NP_001121681.1); staphylococcal nuclease and tudor domain containing 1 (SND1; NP_055205.2), ADAM metallopeptidase domain 9 (ADAM9; NP_003807.1); and ezrin (EZR; NP_003370.2). Thus the EVs can comprise specific proteins comprising one, two, three, four, five, six, or all seven of TXN, EFEMP1, (GSTM1, SGCD, SND1, ADAM9, and EZR. Without wishing to be bound by theory, it is believed that these proteins provide a therapeutic effect. A summary and diagram of exemplary EVs is shown in FIG. 2. Antibody-directed targeting of EVs increases specificity of delivery of neuroprotective proteins, miRNA and other molecular cargo in the EVs to photoreceptors. This can be used to promote delivery of appropriate concentrations of neuroprotective cargo and internalization by photoreceptors to activate restorative and regenerative processes. Methods for targeting of EVs for delivery of therapeutic agents has been successfully demonstrated in non-ocular tissues (see, e.g., Chen et al., Front. Cell Dev. Biol., 8 Oct. 2021. doi.org/10.3389/fcell.2021.751079.

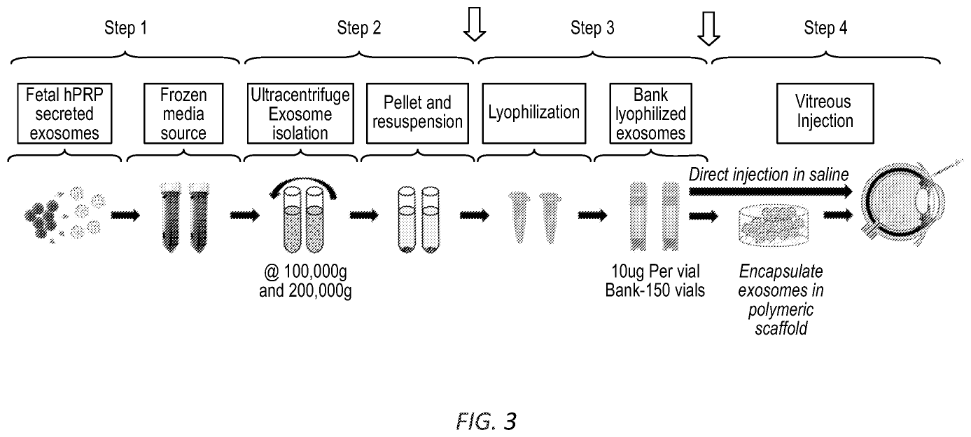

hPRP cells secrete EVs into the culture media continuously, e.g., throughout the entire manufacturing and culturing process. The EVs can be obtained from the hPRP cells using known methods. An exemplary manufacturing method is summarized in FIG. 3. In this example, EVs secreted by hPRP cells are obtained from their culture media. Media is frozen and then ultracentrifuge to isolate EVs and discard debris, dead cells, and other undesirable components. The final pellet is resuspended in PBS, lyophilized and banked, keeping the EVs structure intact. Finally, the EVs are resuspended in an aqueous carrier (optionally comprising PBS or a hydrogel carrier) and injected in the vitreous of the eye. The media comprises the selected and secreted EVs.

The EVs or hPRPs can be suspended, e.g., in a physiologically acceptable buffer such as phosphate buffer saline (PBS), or in a biocompatible polymer, e.g., a hydrogel. Since EVs injected simply in Phosphate buffer saline (PBS) may be targeted by the host immune system, thereby reducing their efficacy in inducing regeneration, polymeric scaffolds comprising biocompatible biomaterials can be used to encapsulate the EVs before delivery: the hPRPs can also be formulated in a polymeric gel for administration. Suitable bio-degradable and biocompatible polymeric materials can include one or more of gelatin, chondroitin sulphate, hyaluronic acid, alginate, collagen and chitosan-based scaffolds, e.g., gelatin-hyaluronic acid gel, e.g., a composition comprising gelatin hydroxyphenylpropionic acid (gelatin-HPA) and hyaluronic acid-tyramine (HA-Tyr) (see, e.g., WO2021113515 and Dromel et al., NPJ Regen Med. 2021 Dec. 20; 6(1):85). Such materials offer non-toxic and biocompatible degradation products while themselves triggering a higher immune response than synthetic polymer. Other materials that can be used includes but not limited to polycaprolactone (PCL), polylactic-gly colic acid (PLGA), polyethylene glycol (PEG), polylactic acid (PLA), polyetherimide (PEI), PNIPAAM, or hydroxy ethyl-methacrylate. See, e.g., US 20140231381 and US 20110004304.

### Methods of Use

The EVs and hPRP produced as described herein can be used to treat retinal degenerative diseases, e.g., diseases associated with loss of photoreceptor rod or cone cells such as macular degeneration, e.g., dry macular degeneration; retinitis pigmentosa; and other Inherited Retinal Diseases, including Stargardt's Disease, Leber congenital amaurosis (LCA), or cone-rod dystrophy (CRD); in particular, the present methods can be used in conditions where rods are lost first. For example, certain retinal diseases affected by loss of cones (e.g., secondary loss of cones in retinitis pigmentosa (RP) leads to blindness) can be treated using hPRPs or EVs isolated from hPRP cultures as described herein; in some embodiments the EVs comprise rod-derived cone viability factor (RdCVF), an inactive thioredoxin secreted by rod photoreceptors that protects cones from degeneration (Aft-Ali et al., Cell. 2015 May 7; 161(4):817-32). Degeneration of retinal photoreceptors is also seen in the late stage of dry AMD, also known as geographic atrophy (GA), In some embodiments, the present methods can include administering a population of hPRPs to replace cells lost to degenerative disease. The inherited retinal diseases treatable by a method described herein include Bardet-Biedl syndrome, autosomal recessive; Chorioretinal atrophy or degeneration, autosomal dominant; Cone or cone-rod dystrophy, autosomal dominant; Cone or cone-rod dystrophy, autosomal recessive; Cone or cone-rod dystrophy, X-linked; Congenital stationary night blindness, autosomal dominant; Congenital stationary night blindness, autosomal recessive: Congenital stationary night blindness, X-linked; Leber congenital amaurosis, autosomal dominant; Leber congenital amaurosis, autosomal recessive; Macular degeneration, autosomal dominant; Macular degeneration, autosomal recessive; Ocular-retinal developmental disease, autosomal dominant: Optic atrophy, autosomal dominant; Optic atrophy, autosomal recessive; Optic atrophy, X-linked; Retinitis pigmentosa, autosomal dominant; Retinitis pigmentosa, autosomal recessive; Retinitis pigmentosa, X-linked; X-linked; Usher syndrome, autosomal recessive; autosomal dominant retinopathy; autosomal recessive retinopathy; mitochondrial retinopathy, and X-linked retinopathy, as well as retinopathy associated with syndromic/systemic diseases. See, e.g., RetNet, the Retinal Information Network, available at sph.uth.edu/retnet/home.htm.

The methods generally include administering therapeutically effective amounts of hPRPs or EVs as described herein, e.g., by intraocular administration, e.g. by subconjunctival, intracameral, or intravitreal injection: see, e.g., Amo et al., Prog Retin Eye Res. 2017 March; 57:134-185; Yamada and Olsen, Dev Ophthalmol. 2016:55:71-83. For example, purified and lyophilized EVs can be stored at −80° C., and can be resuspended, e.g., in the operating room, before administration, e.g., by vitreous injection, e.g., with a 31-gauge needle. The EVs can be resuspended in a physiologically acceptable buffer, e.g., PBS, or in a polymeric scaffold as described herein. Dissociated hPRP cells, e.g., in suspension, in a hydrogel (e.g., as described herein, e.g., a gelatin-hyaluronic acid gel, e.g., a composition comprising gelatin hydroxyphenylpropionic acid (gelatin-HPA) and hyaluronic acid-tyramine (HA-Tyr) (see, e.g., WO2021113515 and Dromel et al., NPJ Regen Med. 2021 Dec. 20; 6(1):85), or in a retrievable permeable capsule loaded with stein cells, can be delivered, e.g., through the retina after pars plana vitrectomy (PPV) or through a transscleral approach without vitrectomy (Hinkle et al., Stem Cell Research & Therapy volume 12, Article number: 538 (2021); Falkner-Radler et al., The British journal of ophthalmology. 2011; 95(3):370-5; Bhattacharya et al., Curr Mol Biol Rep. 2017 September; 3(3): 172-182.

Approximately 3 ug of lyophilized EVs were delivered in 3 uL per injection in rodent studies. Dosing concentration, quantity, and frequency to be used clinically can be determined based on animal and clinical trial data.

### EXAMPLES

The invention is further described in the following examples, which do not limit the scope of the invention described in the claims.

### Materials and Methods

The following materials and methods were used in the Examples below.

### Cell Culture and Isolation Process of Rods

Hunan Fetus arrival: Fetal eyes arrive within 24 h in 2-degree box. Dissection was started directly in order to keep high viability.

Dissection and dissociation: Dissection of the retina was performed on all eyes. Retinas were then dissociated in papain for 30 min in the incubator. Cell suspension was then centrifuged and seeded in a T25 or T75 flask coated with fibronectin (depending on the number of cells).

Culture for 1-2 weeks: Cells were cultured in a 2D layer for 1-2 weeks using FDA approved media (no animal products; described below) in hypoxic conditions (5% O₂). Upon reaching confluence (about 10 million cells) cells were then passaged and centrifuged to start sorting.

Sort with CD73/CD276 (PE and APC): Cells were stained with CD73-PE and CD276-APC for 30 min in 2° C. After washing and preparation of the cell sorter, cells were sorted by gating about 20-30% of the positive population, Pre-sort and post-sort analyses were performed.

Culture to reach high yield: The final cell line was cultured for 4 weeks with the same media and flasks to obtain about 40 million cells for use in experiments or to create a cell bank.

### Culture Media

DMEM/F12—500 ml
Knockout serum (KSR)—55 ml-10%
100×1-Glutamax 5.5 ml-1%
100× non-essential amino acid—5.5 ml-1%
Sodium Pyruvate—5 ml-1%
B-mercaptoethanol—5 μl-0.001%
rhEGF (peprotech)—1000 ul (10 μg/1 ml)-0.2%
rhFGF (peprotech)—500 ul (10 μg/ml)-0.1%

### Eyecup Collection

1. Eye globe was placed in buffer (HBSS/PBS) on a petri-dish and retina was carefully teased out by removing the lens and vitreous fluid and without trace of RPE or ciliary body.
2. The retina was suspended in 10 ml papain solution for 30 mins at 37° C. to dissociate tissue into single cell suspension.
3. 20 ml of IBSS was added to the tube and the sample was centrifuged at 2000 rpm for 5 min.
4. The buffer was aspirated and the pellet re-suspended in 1 ml media, then plated on a T675 flask for further expansion

### Preparing Cells for Sorting

5. Cells were lifted from confluent flask using trypsin (1:6 in IBSS).
6. Cells were the incubated with trypsin at 37° C. for 3 to 4 min.
7. Cells were collected in 50 ml tubes and around 40 ml media containing KSR was added to the cell suspension.
8. The suspension was centrifuged at 1200 rpm for 5 min at 15° C.
9. Supernatant was aspirated and the cell pellet resuspended in 200 μl of Miltenyi running buffer (cat no. 130-107-207).

### Staining for Pure Rod Photoreceptors Sort

10. Cells were incubated in CD73 PE (Cat no #130-095-183, Miltenyi), CD276 Antibody, anti-human, VioBlue® (Cat no #130-099-998) for 30 mins in ice.
11. After incubation the cells were re-suspended in 10 ml of HBSS and filter using 30 um filter (Miltenyi).
12. The sample was then centrifuged at 300 g for 5 mins to wash off the antibody,
13. Meanwhile the TYTO cartridge was prepared by injecting filtered 1 ml TYTO buffer in the input chamber. The cartridge was pressurized to move buffer into the positive chamber and the negative chamber.
14. The buffer was removed from the input chamber to conclude cartridge prep.
15. Supernatant was then discarded and depending upon cell number cells were re-suspended in 5 to 10 ml (4 to 6 million cells in 5 ml and above 5 million in 10 ml) of TYTO buffer and injected into the input chamber of TYTO cartridge for sorting.
16. 100 μl of the cell suspension was placed in an Eppendorf for MACSQUANT analysis before running TYTO sorting.

### Culturing Post-Sorting

1. Using a fine tip pipet, cells in positive chamber were removed and chamber was flushed using HBSS.
2. 50 ul of the cell suspension was put in an Eppendorf for MACSQUANT analysis post-sorting.
3. The rest of the cell suspension was washed with 15 ml of HBSS by centrifuging at 300× for 5 mins.
4. Cell pellet was resuspended in culture media and plated on fibronectin coated T-25 or T-75 flask
5. Incubate at 37° C. in hypoxic conditions (5% O₂).
6. Flask is maintained without media change for 48 hrs
7. 2 days post incubation change complete media.
8. Thereafter every 2 days fresh media was added until confluency was obtained
9. Confluent flasks (should be confluent in 7 to 10 days) were trypsinized and the cells were seeded on one t-75 for further expansion.
10. Media was changed every day until confluency was reached, in 3 to 4 days.

### Cell Sorting and Analysis

### Sorting Strategy

Cells analyzed with MACSQUANT before running the TYTO are set up as the control for running the sort. Markers of choice, CD73 and CD276 are analyzed with the MACSQUANT by tuning the voltages in order to put the desired population in the highest quadrant of the intensity. After this first gating strategy is performed, the TYTO cartridge is placed in the machine and the microfluidic flow is started. Cells are first pushed through the negative chambers in order to stabilize the pressure (around 150 hPa) but also the flow of cells in front of the lasers (40 ms between two cells). In order for the machine to know its velocity, a cell must be stained in at least two different fluorochrome, this way the machine can calculate the time it takes the cell to go from the first laser to the second one, hence measuring its velocity. As soon as both these variables are stable, lasers can be started, and fluorescent data starts to appear showing an approximate of 3000-5000 cells that are being pushed in front of the lasers. Each cell passes in front of the three lasers and its fluorescent intensity is measured and reported on the graph.

The same gating strategy that was used on the control on the MACSQUANT was reported on the live TYTO sorting machine and the sort was started. However, in order to properly capture each cell, the valve speed and delay of action must be properly set up. Back scatter was fitted for each cell population (BSB, and BSV) flowing through the microfluidic device, and threshold was set up at 10\hat{ }2-5.10\hat{ }2 for both BSB and BSV. Threshold and gate channels for PE and VioBlue were centered on the highly fluorescent population, around 10\hat{ }2 to 10\hat{ }2. Gating time was set up at ms with a pressure of 150 hPa. These settings can be altered depending on cell size, shape, granularity, intensity, stiffness, and concentration, which can be changed by analyzing the cell staining and population prior to starting the sort. Overall, the goal is to modify the parameters to obtain a high fluorescent population around 10\hat{ }2 to 10\hat{ }3 in both PE and VioBlue laser that can be sorted.

During the entire sorting process (usually about 2 hours) the percent of sorted, gated and positive cells was measured and reported as a function of time. At the end of the sort, the cartridge was removed from the machine and cells taken back to the cell culture for next steps.

### Flow Cytometry

hPRPs [(5×10\hat{ }5/mL in media) were trypsinized and cell pellet collected was processed for the phenotype then analyzed using a Flow Cytometry assay.

Flow Cytometry was performed using MACSQUANT flow cytometer (Miltenyi, San Diego) (100). Cells collected and fixed with paraformaldehyde at 4° C. for 15 min. Cells were then washed in wash buffer (BD Biosciences) and incubated, at room temperature, in block buffer (Pharmingen staining buffer with 2% goat serum) for 30 min. Blocked cells were seeded onto a flat bottom 96-well plate (treated, sterile, polystyrene, Thomas Scientific) and stained with conjugated primary antibodies (DAPI-Vioblue, CD73-PE. CD276-APC, Cone Arrestin-FITC, Blue opsin-FITC, Rhodopsin-FITC, CRX-APC, Recoverin-APC, Calbindin-FITC, PkCa-FITC, Bma3a-FITC, Ki67-APC, Thy1.2-APC, Vimentin-FITC, Cynoxin-APC, PAX6-APC, NRL-APC, Cmyc-FITC) overnight at room temperature. Primary antibodies were diluted in 200 μL of antibody buffer (TBS, 0.3% Triton X-100 and 1% goat serum). After cells were washed three times for 15 min, secondary antibodies were goat-derived anti-rabbit and anti-mouse and diluted 1:200 in antibody buffer (Jackson Immunoresearch Laboratory). Secondary antibodies were applied and left at room temperature for 3 h. light scatter and fluorescence signals from each well were measured using the MACSQUANT flow cytometer (2×10\hat{ }5 events were recorded). The results were analyzed using the MACSQUAiNTify software (Miltenyi), For each primary antibody DAPI-positive single cell population was gated. The ratio of positive cells in the gated population was estimated in comparison with blank and species-specific isotype control.

### Immunohistochemistry (IHC):

Live hPRPs grown in chamber slides and cryosection from Long Evans left eye were fixed with 4% paraformaldehyde in 0.1 M PBS (Irvine Scientific) at room temperature for 20 min. These fixed cells and sections were blocked and permeabilized with a blocking solution [(Tris-buffered saline (TBS), 0.3% Triton X-100 and 3% goat serum (Jackson ImmunoResearch Laboratories, West Grove, PA)] for 15 min. Samples were then rinsed twice with 0.1 M TBS buffer for 15 min each time, mounted on polysine microscope slides and incubated with primary antibodies overnight at 4° C. (DAPI-Vioblue, CD73-APC, CD276-Vioblue, Rhodpsin-Cy3, Recoverin-FITC, PKCa-Cy3, CRX-APC, Ki67-CV3, PAX6-FITC) at concentrations determined in laboratory. The next day, samples were rinsed three times with TBS for 15 min. Secondary antibodies (goat-derived anti-mouse and anti-rabbit) were applied for 1 h at room temperature. Samples were then washed one last time with TBS before being mounted on polysine microscope slide with 10× viscosity slide mounting medium. Digital images were obtained with an Epifluorescent microscope using 20× objective. Electronic image files were managed using Matlab software.

### In Vivo Assessment of Cell and EV Effect

### In-Vivo Transplantation

Fifteen rd-1 (C3H/HeOuJ) (age 3 weeks, approximate weight 15 g) were used as recipients in the experiment. Transplantation was performed on non-immuno-suppressant mice. Mice were sedated intraperitoneal injection of ketamine (100-200 mg/kg) and xyalzine (20 mg/kg) for anesthesia Eyes were first anesthetized using topical ophthalmic proparacaine (0.5%) followed by Genteal to keep the lens moist during the surgery.

Recipient mice were injected in sub-retinal space and intravitreal with hPRP or Rod-Exo single-cells injections. A conjunctival incision and a small sclerotomy were performed using a fine disposal scalpel. Cells were injected into the subretinal space using a glass pipette (internal diameter, 150 um) attached to a 50-uL Hamilton syringe via a polyethylene tubing. The hPRPs were injected into the retina bleb as a single-cell suspension in PBS. All samples contained approximately 1×10\hat{ }5 cells and the injection volume were 2 uL for all replicates. Similarly, intravitreal injection was performed using 31 g needle with 3 μL of cells in PBS. For sub-retinal injections using a glass coverslip applied on the eye checked bleb presence. Subretinal space injection was considered successful is a shining bleb was seen under the dissection surgical microscope. Triple antibiotic (Bac/Neo/Poly) was given locally at the end of the surgery to prevent further infection. The mice were then placed in their cages for a 21 day study.

The research protocol was reviewed and approved by the Schepens Eye Research Institute Animal Facility and was in accordance with the Association for Research in Vision Ophthalmology Statement for the Use of Animals in Ophthalmic and Vision Research.

### Tissue Processing

8-, 15- and 21-Days post transplantation rats were sacrificed by CO₂ suffocation for 2 min. Eyes were enucleated and placed in 4% paraformaldehyde for 24 hrs. Tissues were subsequently saturated with increase concentration of sucrose (5%, 10%, 20%) containing Sorensen phosphate buffer. Eyes were left in 30% sucrose overnight or until dissection. The tissues were embedded in cryo-section gelatin medium overnight and sectioned at 15 μm thickness on a cryostat.

For subretinal injection, during the sectioning process, every 5^{th} section was stained and examined by epi-fluorescence for hPRPs presence with TRA-1-85-FITC and STEM121-FITC (human cells marker), rhodopsin-PE and recoverin-APC (host photoreceptor marker) and DAPI-Vioblue (cell nuclei). Sections were observed under Lecia Sp8 confocal microscope for engraftment and cells survival in both sub-retinal and intravitreal space of the mice.

For vitreous injection, during the sectioning process, every 6^{th} section was stained with H&E and examined under brightfield microscopy for morphology and architecture of the retina, along with photoreceptor layer thickness, Outer nuclear layer of photoreceptor was counted blindly to measure the potential neuroprotective effect of hPRPs. Frozen vials of media are kept at −80 C before use. Before performing isolation and lyophilization of EVs, frozen vials of media were analyzed by Dr. Redenti laboratory, where full proteomics and miRNA analysis was performed. Common protocol for EVs isolation and analysis was performed.

### Rod-Exo Manufacturing Process

The manufacturing process is summarized in FIG. 3. EVs secreted by hPRP cells were obtained from their culture media. Media was frozen and then ultracentrifuged to isolate EVs and discard debris, dead cells, and other undesirable components. The final pellet was resuspended in PBS, lyophilized and banked, keeping the EVs structure intact. Finally. EVs were resuspended in water and injected in the vitreous of the eye. The hPRP secreted EVs throughout the entire manufacturing and culturing process. These EVs were released in the culture media. We saved and froze around 40-50 mL tube of this media after immunoselection (sort), and after passage 2. Those vials of media contain the selected and secreted EVs.

Rod-Exo Isolation from Media

Frozen media from fetal hPRP culture (for fetal-derived EVs) was thawed in a water bath (37 C). Following is a succession of ultracentrifugation of samples. First samples were spun at 300 g for 10 minutes in order to discard cells from the source. Following was a 2000 g spin for 10 minutes to discard dead cells and then a 10,000 g spin for 30 minutes to discard all cell debris. Finally, a double ultracentrifugation was performed to pellet down the EVs (only remaining component of the supernatant): both at 150,000 g for 70 minutes. Final supernatant was finally discarded, and a pellet of EVs was obtained. EVs were then measured, analyzed and used for testing and experiments.

Rod-Exo lyophilization and Preservation

Isolated pelleted EVs were resuspended in a final volume with PBS (50 mL). Lyophilization, by controlled freeze-drying at −80° C., was performed to obtain a final powdered product. This final powder was stored at −80° C. and was banked in vials containing 1 g of powdered EVs.

### Testing of EVs and Proteins Functionality

Lyophilized EVs were resuspended in PBS and tested with Western blotting, Bradford assay, Nano sight, proteomics analysis and ELISA to check for concentration of proteins, of EVs, proteins functionality and presence of therapeutic proteins. Testing and analysis was compared to pre-lyophilized EVs using Western blotting for imaging and analysis of the presence of specific proteins; NANO SIGHT was used for analyzing and measuring the concentration of EVs; Bradford assays were used for measuring the total protein concentration: ELISAs were used for measurement of protein functionality; and proteomics analysis was used for a full analysis of fold change in proteins.

### In-Vivo Testing

In the present experiments (Example 3), seven mice with the genotype rho−/−, aged three weeks and weighing approximately 120 g, along with P23 homozygous mice, aged four weeks and weighing approximately 117 g. were utilized. The mice were not given any immunosuppressant drugs before transplantation. To induce anesthesia, the rice were injected with ketamine (40=80 mg/kg) and xyalzine (10 mg/kg). Proparacaine (0.5%) was topically applied to anesthetize the eyes, followed by Genteal to maintain the moisture of the lens during surgery. Recipient mice were injected in intravitreal space with HPRP (human progenitor rod photoreceptors), gel+HPRP and sham. Gel described is composed of gelatin-hyaluronic acid functionalized with tyramine side chains used at 5.5% (w/v) concentration (Dromel et al., NPJ Regen Med. 2021 Dec. 20; 6(1):85). The polymers were crosslinked using 0.1 U/ml of HRP and 1 mM of H₂O₂Cells and gel were injected into the vitreal cavity using a glass pipette (internal diameter, 100 um) attached to a 50-uL Hamilton syringe via a polyethylene tubing. All samples contained approximately 75,000 cells and the gel injection volume was 1 uL for all replicates. Triple antibiotic (Bac/Neo/Poly) was given locally at the end of the surgery to prevent further infection. The mice were then placed in their cages and returned to their normal habitat. The research protocol was reviewed and approved by the Schepens Eye Research Institute Animal Facility and was in accordance with the Association for Research in Vision Ophthalmology Statement for the Use of Animals in Ophthalmic and Vision Research.

### Optometer Testing

The OptoMotry, designed for rodents by Cerebral Mechanics Inc., is used to perform Optokinetic Tracking (OKT) in a non-invasive manner. We placed mice on a platform surrounded by four LCD screens within a light-protected box, and presenting them with visual stimuli via the screens. To assess VA, mice were presented with vertical sine wave gratings of varying spatial frequencies. The gratings are displayed at a fixed distance from the animal, and the smallest spatial frequency that elicits a tracking response is recorded as the VA threshold. VA is measured separately for each eye, with an average testing time of 20 minutes per animal. For contrast sensitivity, tracking movements are identified as slow, steady head movements in the direction of the rotating grating. Spatial frequency thresholds are measured by testing the mice at various spatial frequencies between 0.064 and 0.514 cycles/degree, with an average testing time of 20 minutes per animal. The OptoMotry device uses a proprietary algorithm that adjusts the testing stimuli based on the mouse's tracking reflexes. Contrast thresholds are measured at a spatial frequency of 02 cycles/degree and calculated as the reciprocal of the Michelson contrast. The reciprocal of the contrast threshold is then plotted for analysis.

### Electroretinogram

To prepare for ERG testing, animals were anesthetized, and their pupils were dilated using a 1% Tropicamide eye drop, followed by a drop of 1% Proparacaine on the corneal surface. To prevent dehydration, a drop of Genteal (a corneal lubricant) was applied to the cornea of the untreated eye. In the treated eye, a drop of 0.9% sterile saline was applied on the cornea to allow electrical contact with the gold wire loop recording electrode. A 25-gauge platinum needle was inserted subcutaneously in the forehead to serve as the reference electrode, while another needle was inserted subcutaneously near the tail to serve as the ground electrode. ERG testing was done by series of flash intensities that is produced by a Ganzfeld, which is controlled by the Diagnosys Espion3. Both scotopic (low-light) and photopic (normal-light) responses were tested. During the test, a brief flash of light was presented to the eye, to stimulate the retina. The electrical response generated by the retina was recorded by the gold wire loop electrode on the cornea and transmitted to the Diagnosys Espion3. The response was analyzed to determine the animal's retinal function. This procedure can help diagnose and monitor various retinal diseases and disorders.

### Example 1. Production of hPRP

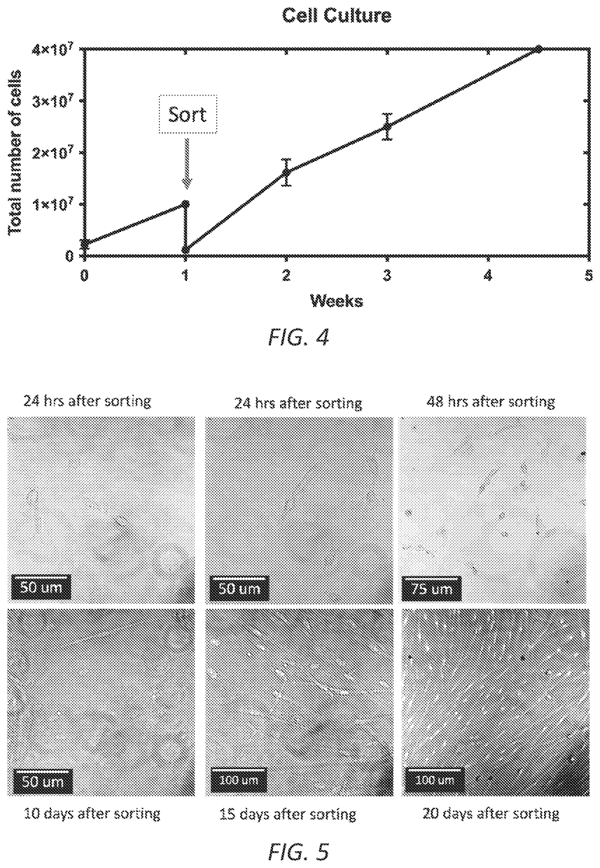

Fetal retinas were cultured as described above in media and sorted based on expression of CD73+ and CD276+ with a Miltenyi TYTO cell sorter. Sorted cell numbers and morphology can be seen in FIGS. 4 and 5.

4.2 Sorting of hPRP from Fetal Tissue

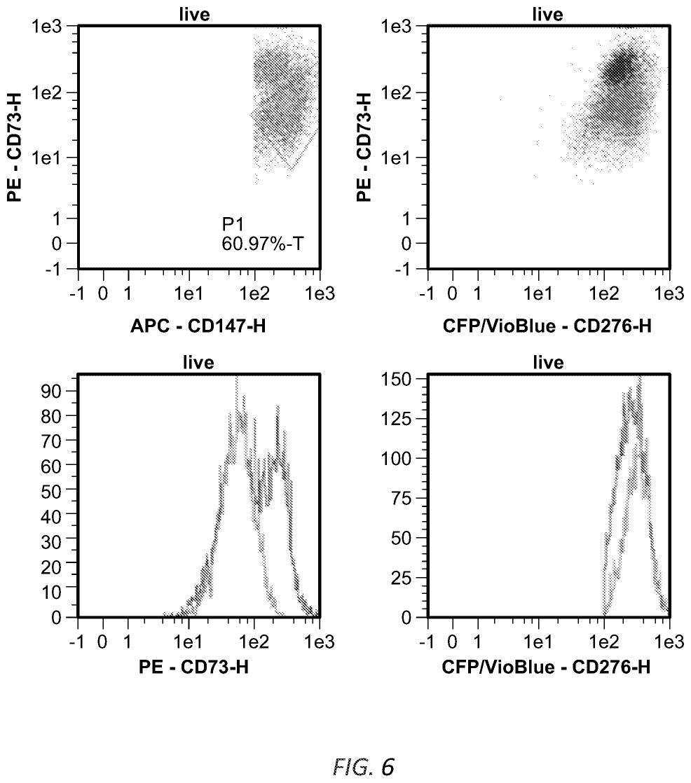

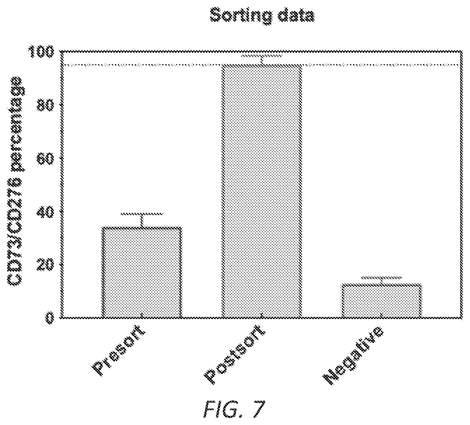

FIG. 6 shows the live gating strategy and sorting of hPRP from fetal retina using both PE and VioBlue laser respectively for CD73 and CD276 positive cells. A gate is drawn around the correct population to start the sort and isolate pure hPRP. Post-sorting, all population are analyzed (input, positive and negative fractions). FIG. shows the percent of positive hPRP in each population, reaching more than 95% in the positive chamber of the TYTO.

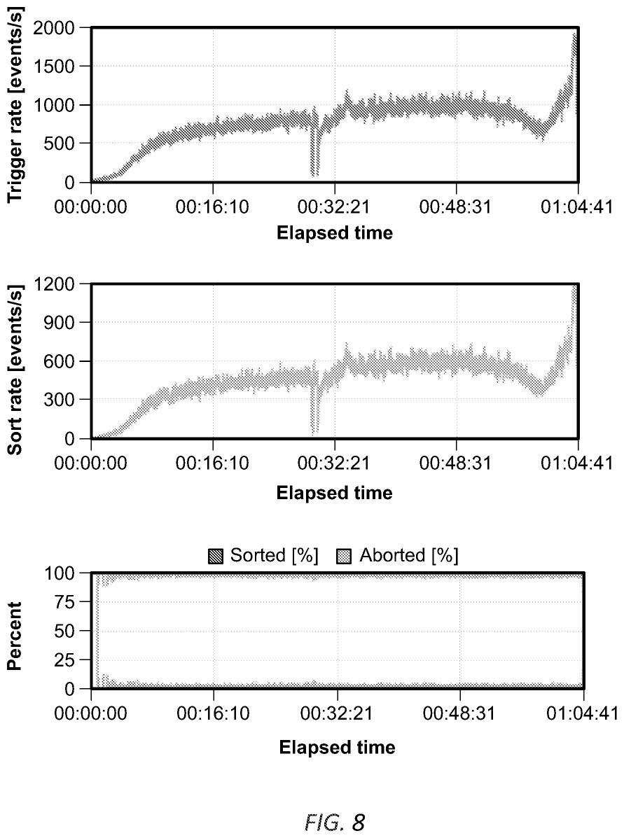

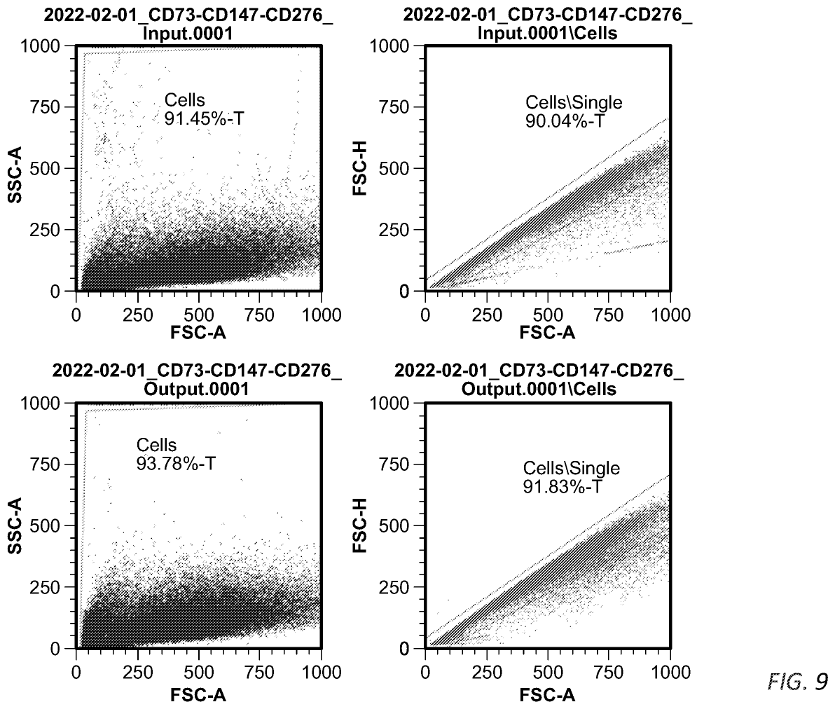

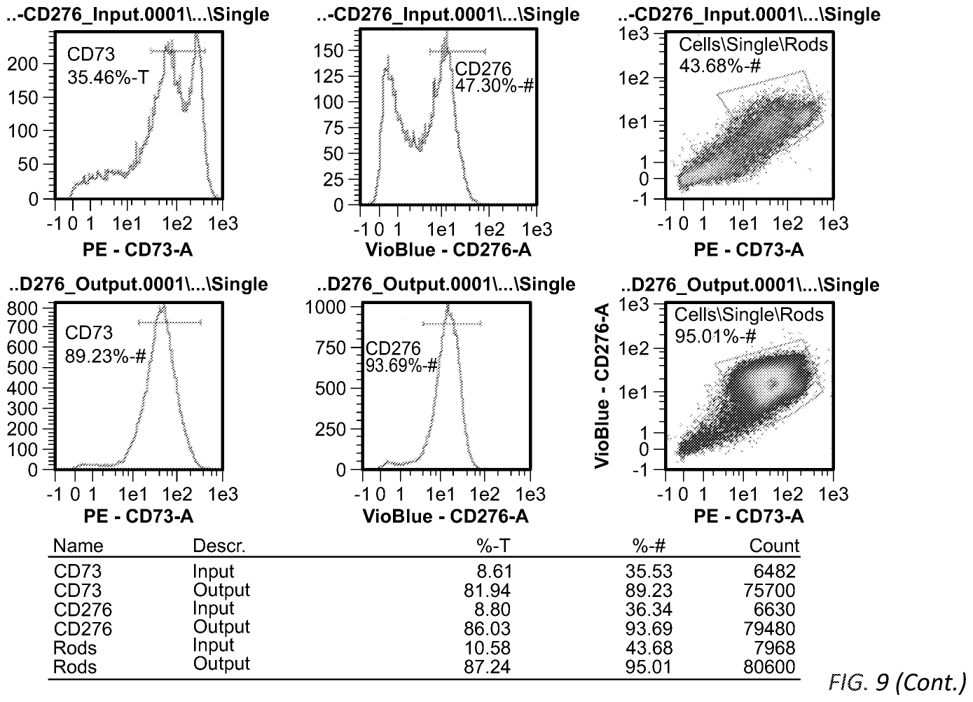

To confirm the success of the entire sort-run, analysis of trigger rate, sort rate, and percent of successfully sorted cells is monitored and analyzed throughout the entire process. FIG. 8. Shows the evolution of those variables during a 1 h sort. Finally, the final population of hPRP is analyzed in the MACSQUANT flow cytometer to precisely measure the number of positive cells (CD73+/CD276+) which were captured in the positive chamber compared to the input of fetal retina (FIG. 9).

4.3 Characterization of hPRP

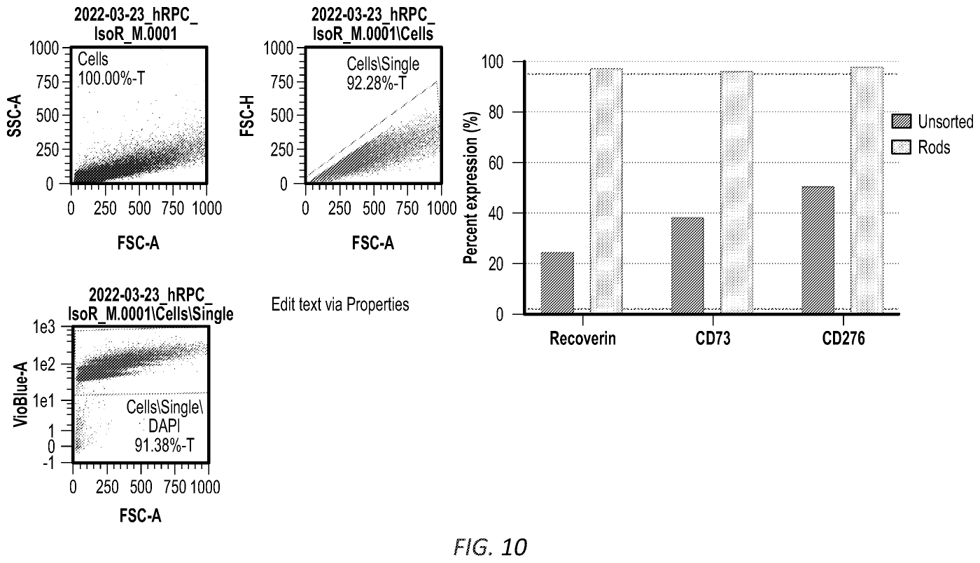

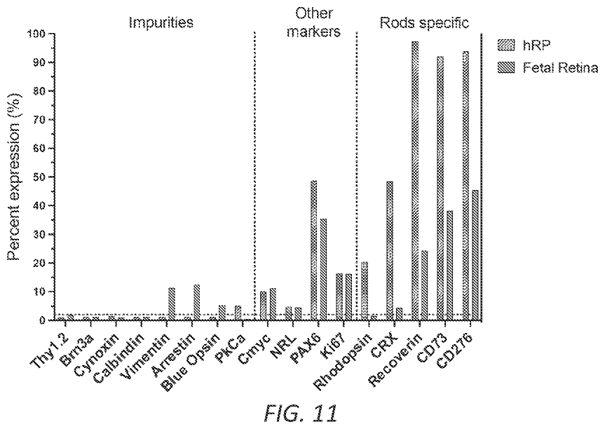

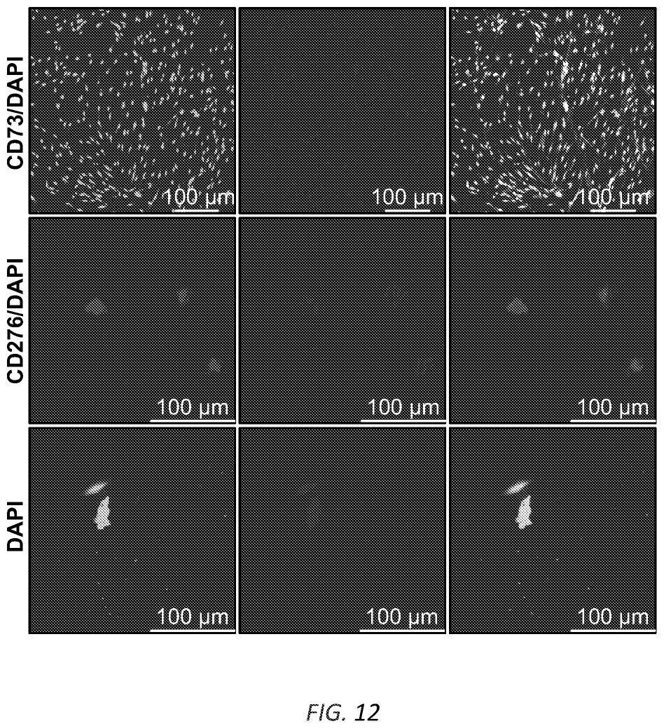

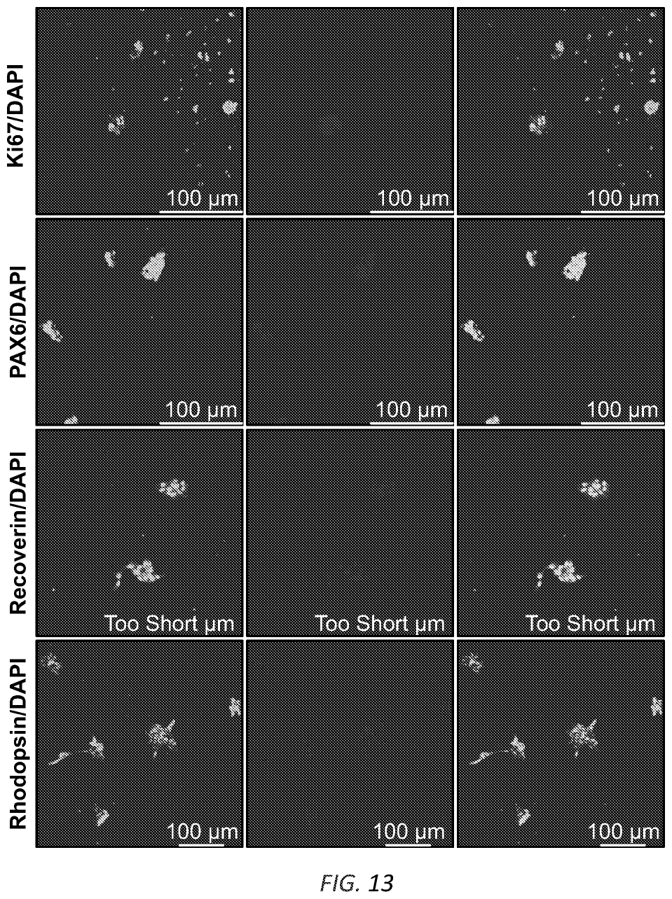

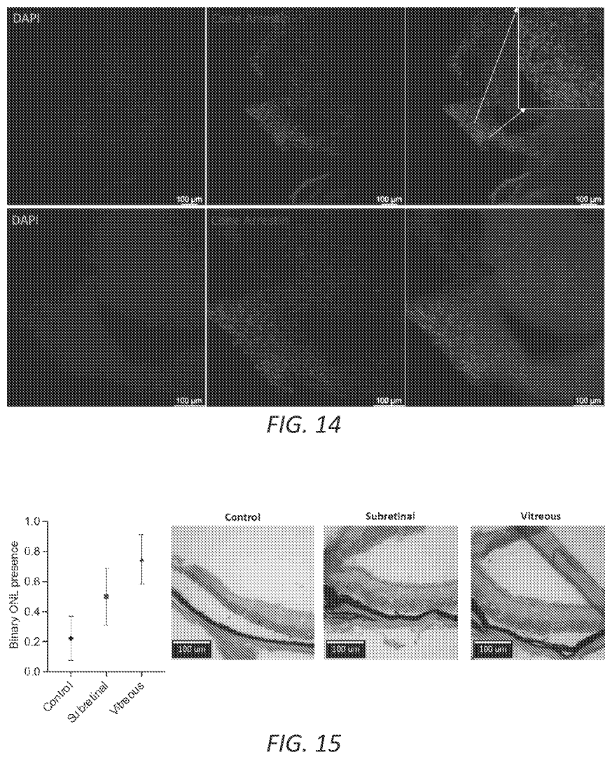

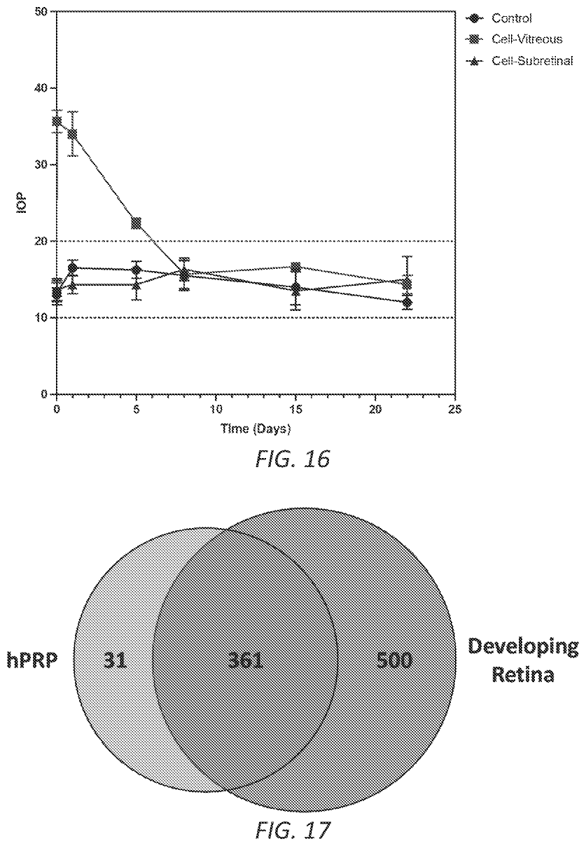

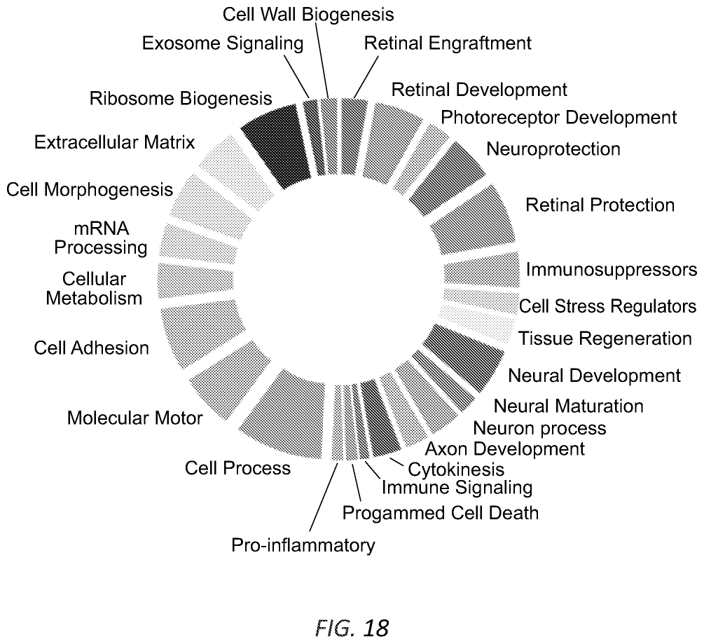

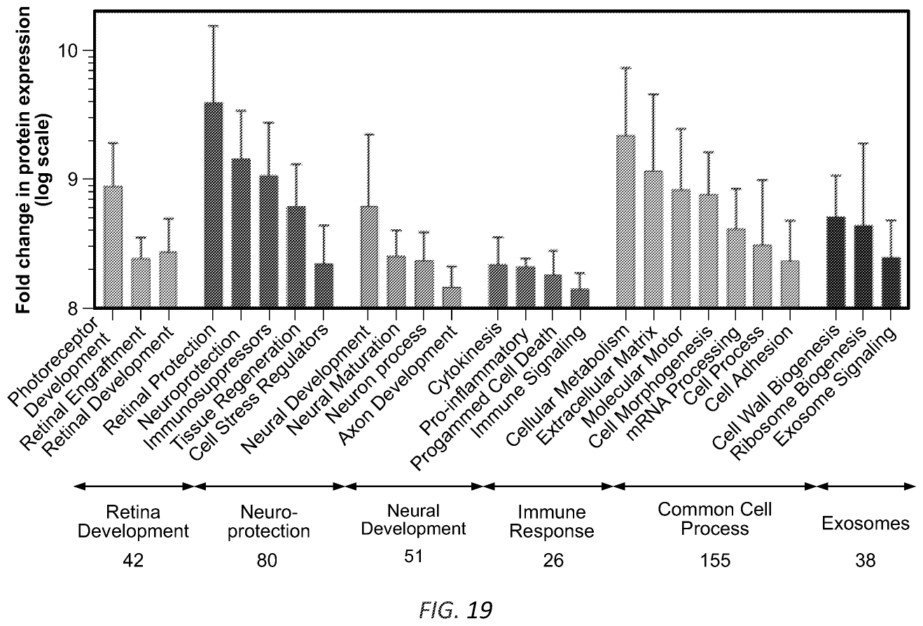

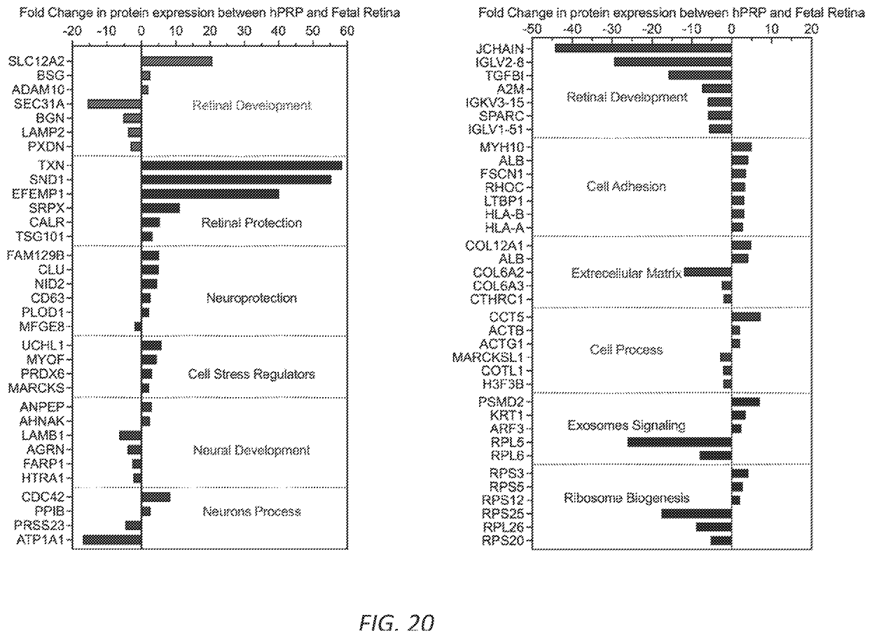

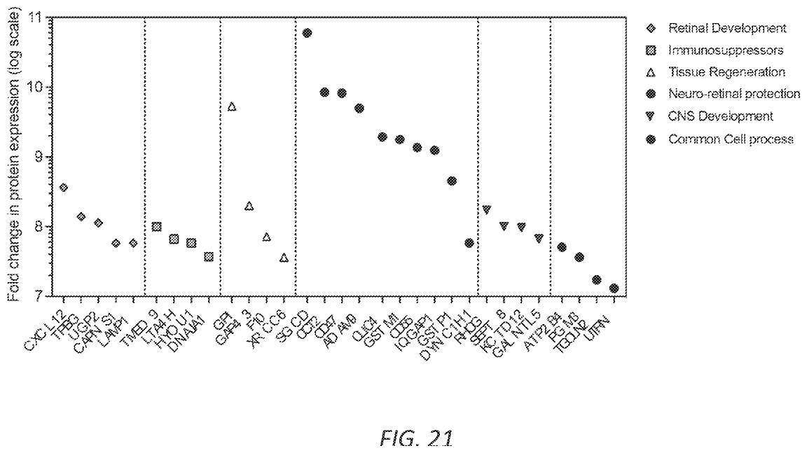

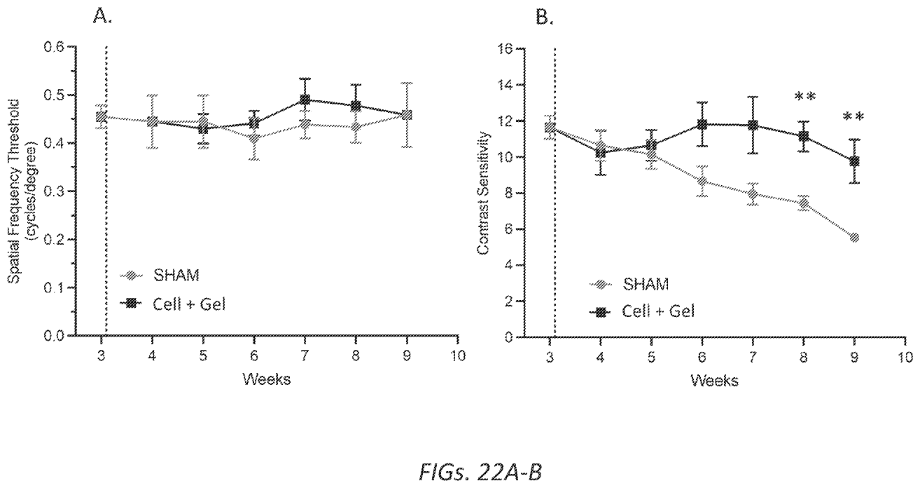

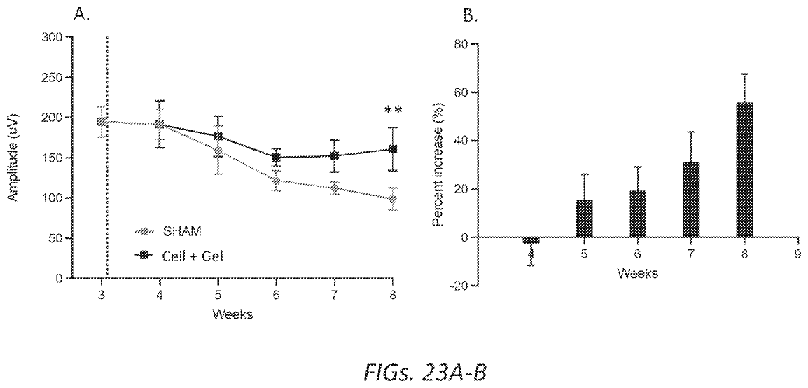

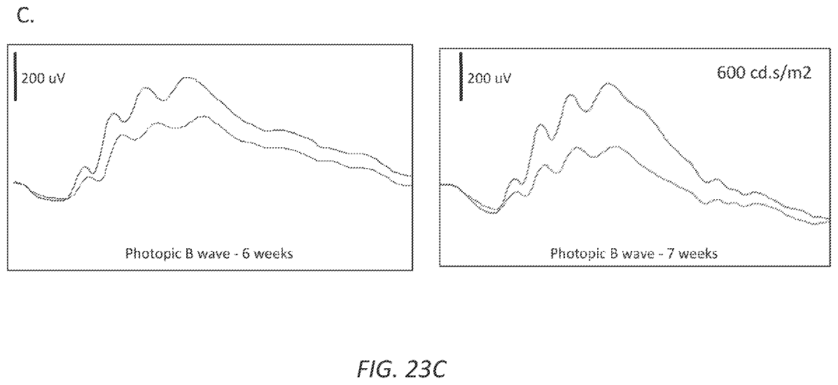

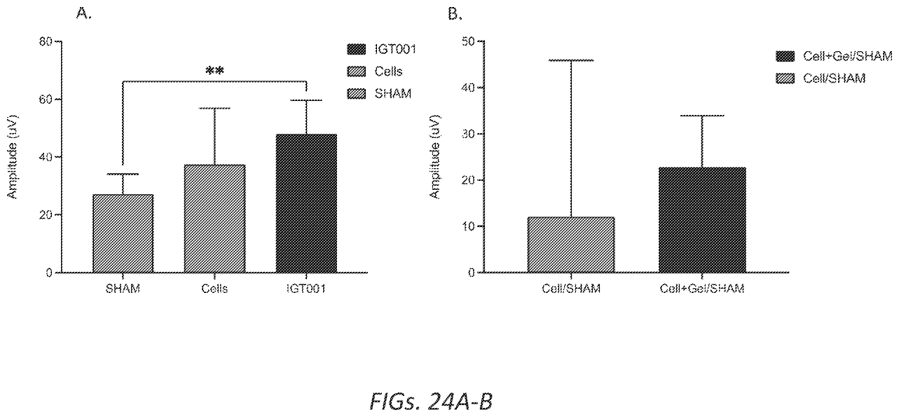

After sorting and 4 weeks of culturing, hPRP were analyzed for their phenotype using both flow cytometry and immune-histochemistry staining (shown in FIGS. 10-13). FIG. 10 presents the critical marker for high purity of rods in hPRP (CD73, CD276 and recoverin). These markers were found to be higher than 95% in all cell population after culturing and sorting. Analysis of many retinal markers were performed to prove the presence of rods and the absences of all other retinal cells with flow cytometry (FIG. 11). The presence of high expression of rod markers confirmed the purity of hRPR, while all other retinal cell markers (RGC, bipolar, amacrine, horizontal, cones, and Muller cell markers) were found to be negative, assuring a high rod purity. To confirm these results, hPRP were stained the same marker and imaged under fluorescent microscopy to prove their expression (FIG. 12-13). High expression of rod and photoreceptor markers were found, while other retinal cell marker were absent from hPRP.

4.4 In Vivo Injection of hPRP In Vitreous and Subretinal Space

To prove the effect of hPRP in neuroprotection of the retina. Cells were injected in the vitreous and subretinal space of rd1 mice (a well-characterized retinal degeneration mouse model). Cells were injected at P25 and animals were sacrificed and analyzed 7 and 21 days post transplantation. To prove neuroprotection, retinas were stained with Cone Arrestin marker (in red) to measure the amount of remaining host cones in the retina (which degenerate at a controlled rate). FIG. 14 shows the wholemount staining of these retinas showing the presence of a large number of cones at day 21, suggesting a high neuroprotection effect from the injection of hPRP. Retinas were also dissected in order to measure the outer nuclear layer (ONL) size, confirming the presence of photoreceptors in the degenerating rd1 mice. After measurement (FIG. 15) a larger number of cells in ONL were found for mice that received hPRP both in the subretinal space and vitreous compared to control. Finally, in order to test the safety of hPRP injection, intraocular pressure (TOP) was measured during the entire experiment, as shown in FIG. 16. A normal increase in IOP was found for a few day s after vitreous injection, and went back to the normal range (10-20 mmHg) after 5 days with no increase for the rest of the experiment.

The sorting process (CD73⁺/CD276⁺) for purification of rod precursor photoreceptor cells yielded a viable 95% pure population of rod photoreceptor cells with a Ki-67 index of about 20% (showing a consistent proliferation).

These cells can be used for cell replacement, drug discovery and screening or other uses where photoreceptors or their precursors might be needed.

### Example 2. Isolation and Characterization of Rod-EVs

Proteomics analysis of EVs was performed and fold change in proteins was compared to 14-weeks fetal retina. As seen in the Venn diagram in FIG. 17, thirty-one proteins were found to be exclusive to our EVs, while 361 were found in our EVS and in fetal retina.

FIG. 18 summarizes the entire proteome cargo found and analyzed in our EVs. Proteins were segregated into six major groups:
1. Retinal Specific: proteins involved in the development and regeneration of retina (cones, photoreceptors, ganglion cells, bipolar cells)
2. Cell protection: proteins involved in neuroprotection of cells
3. CNS development: proteins involved in the development of neural cells (neurons, axons, synapses)
4. Immune response: proteins involved in triggering an immune response and immune reaction. (cytokines, macrophages)
5. Common cell process: proteins found in all cells, involved in normal cell activity (adhesion, proliferation . . . )
6. EVs specific: proteins found on the surface of all EVs

Table 1 shows the major and most important proteins found in each category, with their protein effect, the gene associated to the protein and a description of their activity.

TABLE 1
Vesicle Proteomic Cargo Composition

| | | | |
|---|---|---|---|
| Neuro-retinal | Retinal Protection | TXN | RDCVF secretion |
| Protection | | EFEMP1/GSTM1 | Protects from AMD |
| (Total = 80) | | SRPX/CLIC4/SGCD | Protects from RD/RP |
| | | ADAM9/SND1 | Cone survival factor |
| | | RPN1 | Protects from Stargardt's |
| | | CALR | Protects from CRVO |
| | | CCT2 | Protects from LCA |
| | | IQGAP1 | Protects from CNV |
| | | DYNC1H1 | Rod inner/outer segments |
| | Neuroprotection | MFGE8/CD63 | VEGF/Cell survival |
| | | RALB/FAM129B | Apoptosis suppression |
| | Tissue Regeneration | GAPDH | Regeneration |
| | | CAV1 | Retinal homeostasis |
| Retinal | Photoreceptor | EZR | Rhodopsin |
| Development | Development | GNB4 | Rod development |
| (Total = 42) | | UGP2 | Rods nuclear |
| | | TPBG | Rod bipolar attachment |
| | Retinal Development | GNB1/GNB2 | Opsin |
| | | GNAI2 | Muller opsins |
| | Retinal Engraftment | ITGA5 | Integrin binding |
| | | VCAN | HA binding |
| | | BGN | Chondroitin binding |
| CNS | Neural Development | TENM4 | Synapse genesis |
| Development | | SH3BGRL3 | Neurogenesis |
| (Total = 51) | Neural Process | FGF2 | Growth Factor |
| | | RHOG | Neurites growth |
| | Neural Maturation | GALNTL5 | Axon growth |
| | | SEPT8 | Synapse signaling |
| Immune | Cytokinesis | LBP/EEF1A1 | Cytokine response |
| Response | | IGHM | Antibodies |
| (Total = 26) | Programmed Cell Death | TGFBI | Apoptosis |
| | Immune Signaling | C6/CD99 | Membrane attack complex |
| | | A2MG | CNS stress marker |

In order to simplify the analysis of all proteins, subcategories were created in each of the six different groups established previously. Analysis of effect and activity of each protein was made with a protein database (Uniprot) and a gene database (GeneCard). FIG. 19 shows the fold change of all proteins classified into all the subcategories for the entire proteome cargo of our EVs. Of note is that 42 proteins were found to be for retinal specific development, 80 for cell protection, 51 for CNS development and only 26 for immune response.

The first comparison of proteome cargo was performed between our EVs and the 14-weeks fetal retina EVs. This comparison enables us to characterize the specific beneficial therapeutics but more importantly the immune triggering protein. As seen in FIG. 20, most common cell processes and EVs have a similar expression in the fetal retina and our EVs. On average in retinal specific proteins when comparing fetal retina and our EVs. However, neuroprotective proteins are extremely upregulated in our EVs compared to the fetal retina. Of most importance is the presence of well-known proteins involved in a lot of failure in cell therapy (TGFB1) and immune response (JCHAIN). In the analysis we found that these proteins are expressed 10,000 times less in our EVs compared to the fetal retina. Of note is that other immune triggering proteins were found at same level in the fetal retina and in our EVs.

The final analysis performed in the proteome cargo of our EVs showed the proteins present only in our EVs but absent in the fetal retina. As seen in FIG. 21, 31 proteins were found to be absent in the fetal retina while being highly expressed in our EVs. Of note is that no immune triggering proteins were found to be exclusive to our EVs or absent from the fetal retina, which suggest that those immune triggering proteins are common proteins found in most stem cell therapies.

The total list of proteins found in the EVs (402) and analyzed as described above is provide in Table 2.

TABLE 2
List of all proteins found in hPRP-Exo with fold change

| Protein | GN | Rod-Exo | Protein effect | Protein Category |
|---|---|---|---|---|
| F5GX11 | PSMA1 | 1.10E+08 | Lisosomes | Axon development |
| P09543 | CNP | 1.63E+08 | Axon genesis | Axon development |
| A0A087WYF1 | LAMA2 | 7.55E+07 | Migration | Axon development |
| O14818 | PSMA7 | 8.82E+07 | Lisosomes | Axon development |
| C9JCK5 | PSMA2 | 1.05E+08 | Lisosomes | Axon development |
| G3V3U4 | PSMA6 | 1.43E+08 | Lisosomes | Axon development |
| A0A0A0MTC7 | LAMA4 | 2.36E+08 | Adhesion | Axon development |
| P11047 | LAMC1 | 1.80E+08 | Adhesion | Axon development |
| P07942 | LAMB1 | 2.61E+08 | Migration | Axon development |
| Q96CX2 | KCTD12 | 9.61E+07 | At synapses | Axon development |
| A2BDY9 | HLA-A | 4.48E+07 | Antigen | Cell adhesion |
| G3V511 | LTBP2 | 1.45E+07 | Fibronectin | Cell adhesion |
| H0Y4I7 | HLA-B | 1.43E+08 | Antigen | Cell adhesion |
| A0A087WZU5 | TSPAN6 | 4.65E+07 | cell-cell | Cell adhesion |
| | | | interaction | |
| E7EV71 | LTBP1 | 2.15E+08 | Fibronectin | Cell adhesion |
| Q5SRN7 | HLA-A | 7.40E+07 | Antigen | Cell adhesion |
| P35556 | FBN2 | 3.92E+07 | Binding | Cell adhesion |
| P18084 | ITGB5 | 1.59E+08 | Integrin | Cell adhesion |
| P35555 | FBN1 | 3.92E+07 | Binding | Cell adhesion |
| H3BQF7 | IST1 | 1.37E+08 | Cell process | Cell adhesion |
| Q92896 | GLG1 | 1.38E+08 | Binds fibroblast | Cell adhesion |
| E9PSH3 | TSPAN4 | 1.15E+08 | cell-cell | Cell adhesion |
| | | | interaction | |
| K7ENU8 | BCAM | 5.83E+07 | Adhesion | Cell adhesion |
| P60981 | DSTN | 5.83E+07 | Actin binding | Cell adhesion |
| A0A0D9SF54 | SPTAN1 | 1.21E+08 | Cytoskeleton | Cell adhesion |
| Q07065 | CKAP4 | 1.77E+08 | Microtubules | Cell adhesion |
| Q8NG11 | TSPAN14 | 4.69E+08 | cell-cell | Cell adhesion |
| | | | interaction | |
| P21589 | NT5E | 2.26E+08 | Nociception | Cell adhesion |
| A0A2R8Y6L3 | RPS10-NUDT3 | 1.47E+08 | Hydrolase | Cell adhesion |
| P55001 | MFAP2 | 1.50E+08 | Microfibrils | Cell adhesion |
| Q9BUD6 | SPON2 | 2.90E+08 | Cytoskeleton | Cell adhesion |
| H0Y7V4 | DNAH8 | 5.42E+08 | Microtubules | Cell adhesion |
| P11021 | HSPA5 | 6.68E+08 | Proliferation, | Cell adhesion |
| | | | apoptosis | |
| Q16853 | AOC3 | 7.49E+08 | Binding | Cell adhesion |
| A0A3B31ST1 | TSPAN9 | 5.99E+08 | cell-cell | Cell adhesion |
| | | | interaction | |
| P07996 | THBS1 | 8.55E+08 | cell-matrix | Cell adhesion |
| | | | interaction | |
| P46939 | UTRN | 1.31E+07 | ECM adhesion | Cell adhesion |
| A0A087WXX2 | ALDOB | 7.69E+07 | Actin removal | Cell morphogenesis |
| I3L161 | PLSCR3 | 2.14E+08 | Mitochondria | Cell morphogenesis |
| A0A0J9YX77 | MGAM | 1.45E+08 | sucrose | Cell morphogenesis |
| | | | degradation | |
| Q60FE5 | FLNA | 1.87E+08 | Actin | Cell morphogenesis |
| B1AHL2 | FBLN1 | 3.22E+08 | Actin | Cell morphogenesis |
| Q14315 | FLNC | 2.69E+08 | Actin | Cell morphogenesis |
| P23142 | FBLN1 | 2.48E+08 | Actin | Cell morphogenesis |
| P09525 | ANXA4 | 1.89E+08 | Secretion | Cell morphogenesis |
| P11233 | RALA | 9.33E+07 | Cell division | Cell morphogenesis |
| P50995 | ANXA11 | 6.05E+08 | Cytokinesis | Cell morphogenesis |
| P55072 | VCP | 4.21E+08 | Everywhere | Cell morphogenesis |
| Q71U36 | TUBA1A | 9.13E+08 | Microtubule | Cell morphogenesis |
| Q9BUF5 | TUBB6 | 1.27E+09 | Microtubule | Cell morphogenesis |
| Q8WZ42 | TTN | 8.1E+08 | Mitosis | Cell morphogenesis |
| Q13509 | TUBB3 | 1.47E+09 | Microtubule | Cell morphogenesis |
| P08133 | ANXA6 | 2.85E+09 | Secretion | Cell morphogenesis |
| P04350 | TUBB4A | 1.92E+09 | Microtubule | Cell morphogenesis |
| P07437 | TUBB | 2.02E+09 | Microtubule | Cell morphogenesis |
| P68371 | TUBB4B | 2.02E+09 | Microtubule | Cell morphogenesis |
| A0A087WT27 | PGM3 | 3.64E+07 | Nucleotide | Cell morphogenesis |
| Q16658 | FSCN1 | 7.41E+07 | Actin | Cell process |
| P35580 | MYH10 | 1.55E+08 | Migration | Cell process |
| O75083 | WDR1 | 8.24E+07 | Actin binding | Cell process |
| E9PQH6 | RHOC | 1.91E+08 | Support, motor | Cell process |
| H0YJM8 | PSMB5 | 5.88E+07 | Removes protein | Cell process |
| A0A0C4DGB6 | ALB | 2.89E+08 | Binding | Cell process |
| C9JRL4 | MDH1 | 4.38E+07 | Cytosol | Cell process |
| | | | metabolism | |
| P20618 | PSMB1 | 1.03E+08 | Removes protein | Cell process |
| P02794 | FTH1 | 1.50E+08 | Ion statis | Cell process |
| Q99805 | TM9SF2 | 6.64E+07 | Cell process | Cell process |
| F8W1R7 | MYL6 | 1.80E+08 | Migration | Cell process |
| P61225 | RAP2B | 1.01E+08 | Regulator | Cell process |
| A0A494C0U1 | TPP2 | 5.53E+07 | Homeostasis | Cell process |
| Q6P163 | APOC2 | 1.16E+08 | Neutral | Cell process |
| F5H6E2 | MYO1C | 1.29E+08 | Actin binding | Cell process |
| D6RHE2 | EIF4E1B | 3.98E+07 | Ribonucleoprotein | Cell process |
| C9J4V0 | RAB7A | 1.32E+08 | Regulator | Cell process |
| P29373 | CRABP2 | 1.31E+08 | Retinoid | Cell process |
| Q9BS26 | ERP44 | 6.64E+07 | Inside cells | Cell process |
| C9JZR2 | CTNND1 | 9.19E+07 | Actin | Cell process |
| J3KTM9 | KPNB1 | 6.09E+07 | Nuclear pore | Cell process |
| | | | complex | |
| C9IY94 | SEPT2 | 1.77E+08 | Cell adhesion | Cell process |
| P20340 | RAB6A | 2.74E+08 | Regulator | Cell process |
| P28070 | PSMB4 | 1.82E+08 | Removes protein | Cell process |
| P35579 | MYH9 | 3.91E+08 | Migration | Cell process |
| P10301 | RRAS | 8.45E+07 | Actin | Cell process |
| Q7Z304 | MAMDC2 | 2.07E+08 | MAM | Cell process |
| B4DQU5 | RAB11A | 1.67E+08 | Regulator | Cell process |
| P61026 | RAB10 | 2.52E+08 | Regulator | Cell process |
| P61006 | RAB8A | 2.74E+08 | Regulator | Cell process |
| Q14764 | MVP | 5.71E+08 | Vault | Cell process |
| Q8N6Y2 | LRRC17 | 2.99E+08 | Bone marrow | Cell process |
| | | | control | |
| M0R0E8 | ZNF791 | 3.65E+08 | Zinc binding | Cell process |
| Q9H0U4 | RAB1B | 3.32E+08 | Regulator | Cell process |
| F5H265 | UBC | 1.08E+09 | Eucaryotic cell | Cell process |
| P02792 | FTL | 3.57E+08 | Ion statis | Cell process |
| Q5JXB2 | UBE2NL | 4.14E+09 | Protein folding | Cell process |
| P30041 | PRDX6 | 2.43E+08 | Reduce oxidative | Cell stress |
| | | | stress | regulators |
| Q9BSK4 | FEM1A | 5.82E+07 | Anti-inflammatory | Cell stress |
| | | | | regulators |
| O15040 | TECPR2 | 4.50E+07 | Anti-inflammatory | Cell stress |
| | | | | regulators |
| K7ELW0 | PARK7 | 8.21E+07 | Inhibitor | Cell stress |
| | | | | regulators |
| Q9Y625 | GPC6 | 5.83E+07 | Growth factors | Cell stress |
| | | | binding | regulators |
| P30101 | PDIA3 | 2.06E+08 | Stress marker | Cell stress |
| | | | | regulators |
| F5GZS6 | SLC3A2 | 9.69E+07 | Membrane protein | Cell stress |
| | | | | regulators |
| H7C3T4 | PRDX4 | 1.70E+08 | Reduce oxidative | Cell stress |
| | | | stress | regulators |
| A0A024QZ42 | PDCD6 | 2.01E+08 | Stress marker | Cell stress |
| | | | | regulators |
| P61224 | RAP1B | 4.73E+08 | Regulator | Cell stress |
| | | | | regulators |
| A0A0A0MSI0 | PRDX1 | 2.54E+08 | Reduce oxidative | Cell stress |
| | | | stress | regulators |
| H7C5E8 | TF | 6.54E+08 | Process | Cell stress |
| | | | | regulators |
| Q8WUM4 | PDCD6IP | 6.84E+08 | Stress marker | Cell stress |
| | | | | regulators |
| P31689 | DNAJA1 | 3.71E+07 | Apoptosis | Cell stress |
| | | | suppression | regulators |
| J3KTF1 | HYOU1 | 5.83E+07 | Protects oxidative | Cell stress |
| | | | stress | regulators |
| P61204 | ARF3 | 2.09E+08 | Ribosomes | Cell wall Biogenesis |
| P04259 | KRT6B | 2.13E+08 | Epidermal barrier | Cell wall Biogenesis |
| R4GMT0 | ACTR1A | 1.26E+08 | Microtubules | Cell wall Biogenesis |
| P13645 | KRT10 | 2.50E+08 | Epidermal barrier | Cell wall Biogenesis |
| Q9NVM1 | EVA1B | 1.64E+08 | Membrane protein | Cell wall Biogenesis |
| P04264 | KRT10 | 1.20E+09 | Epidermal barrier | Cell wall Biogenesis |
| O00560 | SDCBP | 1.42E+09 | Genesis | Cell wall Biogenesis |
| K7ERI8 | K7ERI8 | 1.24E+08 | ATP | Cellular metabolism |
| P50395 | GDI2 | 1.76E+08 | ADP | Cellular metabolism |
| P01111 | NRAS | 1.02E+08 | GTP | Cellular metabolism |
| P54687 | BCAT1 | 6.03E+07 | CNS | Cellular metabolism |
| Q96FQ6 | S100A16 | 1.29E+08 | Proliferation | Cellular metabolism |
| B5MD04 | PRAME | 4.10E+08 | Cell proliferation | Cellular metabolism |
| M0QYF6 | GPR108 | 2.34E+08 | AAV transduction | Cellular metabolism |
| Q14697 | GANAB | 2.34E+08 | Glycolysis | Cellular metabolism |
| P04075 | ALDOA | 3.91E+08 | Fructose | Cellular metabolism |
| A0A0A0MS51 | GSN | 3.21E+08 | Filaments | Cellular metabolism |
| Q9C0H2 | TTYH3 | 2.00E+08 | Calcium transfer | Cellular metabolism |
| P14618 | PKM | 1.17E+09 | ATP | Cellular metabolism |
| P69891 | HBG1 | 1.50E+10 | Oxygen binding | Cellular metabolism |
| C9JNR5 | INS | 1.45E+10 | Insulin | Cellular metabolism |
| O43493-2 | TGOLN2 | 1.73E+07 | Trans-Golgi | Cellular metabolism |
| I3L2H4 | HGS | 1.18E+07 | Signaling sp | Cytokinesis |
| A0A075B6N9 | IGHM | 8.22E+07 | Antibody | Cytokinesis |
| A0A075B6I0 | IGLV8-61 | 1.34E+08 | Antibody | Cytokinesis |
| O95865 | DDAH2 | 1.77E+08 | Vascularization | Cytokinesis |
| P68104 | EEF1A1 | 4.09E+08 | Cytokine | Cytokinesis |
| B9A064 | IGLL5 | 3.20E+08 | Antibody | Cytokinesis |
| P18428 | LBP | 2.31E+08 | Cytokine response | Cytokinesis |
| A0A0A0MS10 | IGHV2-5 | 4.30E+08 | Anybody | Cytokinesis |
| P01701 | IGLV1-51 | 1.87E+08 | Antibody | Cytokinesis |
| A0A0B4J1T9 | IGKV3-15 | 3.75E+08 | Antibody | Cytokinesis |
| P01709 | IGLV2-8 | 1.18E+08 | Antibody | Cytokinesis |
| D6RD17 | JCHAIN | 2.46E+08 | Antibody | Cytokinesis |
| Q13200 | PSMD2 | 4.98E+08 | Proteosome | Exosome signaling |
| Q5T8U2 | RPL7A | 1.08E+08 | Ribonucleoprotein | Exosome signaling |
| E9PJD9 | RPL27A | 1.37E+08 | Ribonucleoprotein | Exosome signaling |
| B5MCW2 | RPL3 | 1.01E+08 | Ribonucleoprotein | Exosome signaling |
| QST7N0 | RPL5 | 6.05E+07 | Ribonucleoprotein | Exosome signaling |
| Q02878 | RPL6 | 5.84E+08 | Ribonucleoprotein | Exosome signaling |
| H0YA55 | ALB | 8.36E+07 | Binding | Extracellular Matrix |
| E7ENL6 | COL6A3 | 1.62E+07 | Collagen | Extracellular Matrix |
| A0A087X0S5 | COL6A1 | 8.36E+07 | Collagen | Extracellular Matrix |
| P12111 | COL6A3 | 1.16E+08 | Collagen | Extracellular Matrix |
| D6RGG3 | COL12A1 | 4.35E+08 | Collagen | Extracellular Matrix |
| Q15113 | PCOLCE | 8.82E+07 | Collagen binding | Extracellular Matrix |
| P20908 | COL5A1 | 1.28E+08 | Collagen | Extracellular Matrix |
| B4DLR2 | FAP | 1.53E+08 | Degradation | Extracellular Matrix |
| B0V114 | FLOT1 | 1.31E+08 | Scaffold | Extracellular Matrix |
| P03956 | MMP1 | 1.26E+08 | Collagenase | Extracellular Matrix |
| A0A0C4DFX3 | EMILIN1 | 5.22E+08 | Binding | Extracellular Matrix |
| Q76M96 | CCDC80 | 1.06E+08 | Cell adhesion | Extracellular Matrix |
| P02461 | COL3A1 | 2.25E+08 | Collagen | Extracellular Matrix |
| P12110 | COL6A2 | 5.65E+07 | Collagen | Extracellular Matrix |
| Q96CG8 | CTHRC1 | 2.69E+08 | Collagen | Extracellular Matrix |
| P08253 | MMP2 | 6.92E+08 | Degradation | Extracellular Matrix |
| P02452 | COL1A1 | 1.04E+09 | Collagen | Extracellular Matrix |
| A0A087WTA8 | COL1A2 | 3.03E+09 | Collagen | Extracellular Matrix |
| P02751-15 | FN1 | 1.49E+10 | Fibronectin | Extracellular Matrix |
| P14209 | CD99 | 1.08E+08 | T cells | Immune signaling |
| G3XAJ6 | RFTN1 | 9.86E+07 | T and B cells | Immune signaling |
| P13671 | C6 | 1.85E+08 | Attack cell | Immune signaling |
| | | | membrane | |
| P01023 | A2M | 1.74E+08 | CNS marker | Immune signaling |
| P11216 | PYGB | 8.88E+07 | Antioxidant | Immunosuppression |
| E9PNW4 | CD59 | 4.69E+08 | Lower MAC | Immunosuppression |
| P04083 | ANXA1 | 1.17E+09 | Immune system | Immunosuppression |
| P09382 | LGALS1 | 1.20E+09 | T cell death | Immunosuppression |
| P35442 | THBS2 | 4.56E+08 | cell-cell | Immunosuppression |
| | | | interaction | |
| O60814 | HIST1H2BK | 7.44E+08 | Nucleus | Immunosuppression |
| Q08380 | LGALS3BP | 5.36E+09 | T cell death | Immunosuppression |
| P09960 | LTA4H | 6.64E+07 | Protects | Immunosuppression |
| | | | inflammation | |
| Q9BVK6 | TMED9 | 9.98E+07 | Binding for rods | Immunosuppression |
| P50991 | CCT4 | 8.90E+07 | Actin | Molecular motor |
| F8VR50 | ARPC3 | 1.13E+08 | Motility | Molecular motor |
| O15511 | ARPC5 | 4.22E+07 | Motility | Molecular motor |
| P07951 | TPM2 | 1.28E+08 | Actin binding | Molecular motor |
| Q01518 | CAP1 | 1.42E+08 | Actin | Molecular motor |
| E9PK52 | EPB41L2 | 7.91E+07 | Actin Binding | Molecular motor |
| P52907 | CAPZA1 | 9.1E+07 | Actin | Molecular motor |
| P49006 | MARCKSL1 | 2.53E+07 | Actin | Molecular motor |
| O15144 | ARPC2 | 7.71E+07 | Motility | Molecular motor |
| P67936 | TPM4 | 2.22E+08 | Actin binding | Molecular motor |
| E7ENZ3 | CCT5 | 1.15E+09 | Actin | Molecular motor |
| P35221 | CTNNA1 | 1.40E+08 | Actin | Molecular motor |
| X6RJP6 | TAGLN2 | 2.95E+08 | Actin | Molecular motor |
| H0YCU9 | TAGLN | 1.04E+08 | Actin | Molecular motor |
| Q9Y490 | TLN1 | 1.07E+08 | Actin | Molecular motor |
| P12814 | ACTN1 | 2.27E+08 | Actin | Molecular motor |
| P07737 | PFN1 | 6.40E+08 | Actin | Molecular motor |
| P40227 | CCT6A | 1.15E+08 | Actin | Molecular motor |
| E9PP50 | CFL1 | 6.61E+08 | Actin | Molecular motor |
| H3BT58 | COTL1 | 1.18E+08 | Actin | Molecular motor |
| P60709 | ACTB | 5.10E+09 | Actin | Molecular motor |
| P63261 | ACTG1 | 5.10E+09 | Actin | Molecular motor |
| P63267 | ACTG2 | 4.45E+09 | Actin | Molecular motor |
| F8WCJ1 | EIF5A2 | 7.30E+07 | mRNA binding | mRNA Processing |
| H0YH88 | NAP1L1 | 1.59E+08 | Nucleosome | mRNA Processing |
| A6PVH9 | CPNE1 | 1.38E+08 | DNA binding | mRNA Processing |
| P16401 | HIST1H1B | 1.46E+08 | Chromosomes | mRNA Processing |
| P16403 | HIST1H1C | 2.47E+08 | Chromosomes | mRNA Processing |
| Q00610 | CLTC | 3.08E+08 | Cell motor | mRNA Processing |
| H7C4S4 | FXR1 | 2.47E+08 | translation | mRNA Processing |
| P62826 | RAN | 1.76E+08 | transport | mRNA Processing |
| K7BMV3 | H3F3B | 1.66E+08 | Chromosomes | mRNA Processing |
| P62805 | HIST1H4A | 5.74E+08 | DNA binding | mRNA Processing |
| Q16777 | HIST2H2AC | 1.26E+09 | Chromosomes | mRNA Processing |
| Q96KK5 | HIST1H2AH | 1.08E+09 | Chromosomes | mRNA Processing |
| C9J0D1 | H2AFV | 1.17E+09 | Chromosomes | mRNA Processing |
| P23634 | ATP2B4 | 5.10E+07 | Transmembrane | mRNA Processing |
| P26641 | EEF1G | 5.83E+07 | Neural retina | Neural development |
| Q6N022 | TENM4 | 5.86E+07 | Neurons and | Neural development |
| | | | axons | |
| P60903 | S100A10 | 2.26E+08 | Cell | Neural development |
| | | | differentiation | |
| P15144 | ANPEP | 4.64E+08 | Neuropeptide | Neural development |
| Q9H2G4 | TSPYL2 | 7.94E+07 | Neural synapses | Neural development |
| FSH2D0 | C1R | 7.52E+07 | Complement | Neural development |
| | | | protein | |
| Q09666 | AHNAK | 5.82E+08 | Differentiation | Neural development |
| Q9Y4F1 | FARP1 | 5.16E+07 | Dendrites | Neural development |
| P62158 | CALM1 | 3.79E+08 | Neuron migration | Neural development |
| A0A494C0G5 | AGRN | 6.63E+07 | Post-synaptic diff | Neural development |
| O00468 | AGRN | 6.63E+07 | Post-synaptic diff | Neural development |
| Q5H9A7 | TIMP1 | 2.26E+08 | Low proliferation, | Neural development |
| | | | low mitosis | |
| P16035 | TIMP2 | 4.32E+08 | Low proliferation, | Neural development |
| | | | low mitosis | |
| P60174 | TPI1 | 2.55E+08 | Motor neurons | Neural development |
| Q14195 | DPYSL3 | 4.27E+08 | Axon guidance | Neural development |
| R4GN98 | S100A6 | 9.68E+08 | Cell | Neural development |
| | | | differentiation | |
| P31949 | S100A11 | 8.69E+08 | Cell | Neural development |
| | | | differentiation | |
| P07355 | ANXA2 | 7.35E+09 | Growth of neurons | Neural development |
| Q7Z4T8 | GALNTL5 | | Neural | |
| | | | development | |
| C9JV02 | SEPT8 | 1.01E+08 | Axons | Neural development |
| Q9NZN4 | EHD2 | 9.35E+07 | Caveolae | Neural maturation |
| P23284 | PPIB | 3.58E+08 | Neurons and | Neural maturation |
| | | | axons | |
| C9K028 | NME1 | 7.74E+07 | Neurons and | Neural maturation |
| | | | axons | |
| P80723 | BASP1 | 1.83E+08 | End of axons | Neural maturation |
| P18669 | PGAM1 | 3.45E+08 | Motor neuron | Neural maturation |
| H7C3J1 | TSSK4 | 3.16E+08 | Structural integrity | Neural maturation |
| P62937 | PPIA | 4.91E+08 | Neurons and | Neural maturation |
| | | | axons | |
| P84095 | RHOG | 1.73E+08 | neurite growth | Neural maturation |
| P60953 | CDC42 | 1.82E+08 | Cell division | Neuron process |
| A0A087WUF6 | FGF2 | 2.87E+07 | FGF growth factor | Neuron process |
| P20073 | ANXA7 | 1.72E+08 | Synapse release | Neuron process |
| Q04917 | YWHAH | 1.86E+08 | morphogenesis | Neuron process |
| P62258 | YWHAE | 4.05E+08 | morphogenesis | Neuron process |
| P63104 | YWHAZ | 4.44E+08 | Neurogenesis | Neuron process |
| P27348 | YWHAQ | 3.51E+08 | morphogenesis | Neuron process |
| P63000 | RAC1 | 4.74E+08 | synapses, | Neuron process |
| | | | dendrites | |
| P05023 | ATP1A1 | 2.17E+07 | Channel | Neuron process |
| O95084 | PRSS23 | 1.10E+08 | Metabolic | Neuron process |
| Q9P2B2 | PTGFRN | 3.16E+08 | Axon's growth | Neuron process |
| Q92743 | HTRA1 | 2.63E+08 | Metabolic | Neuron process |
| Q5T123 | SH3BGRL3 | 1.10E+08 | Cell formation, | Neuron process |
| | | | neurons | |
| Q96TA1 | FAMI29B | 1.02E+08 | Apoptosis | Neuroprotection |
| | | | suppression | |
| F8VNT9 | CD63 | 2.09E+08 | Cell survival | Neuroprotection |
| Q15084 | PDIA6 | 9.78E+07 | Membrane protein | Neuroprotection |
| P05121 | SERPINE1 | 5.62E+07 | Blood cloth | Neuroprotection |
| P22413 | ENPP1 | 1.43E+08 | Angiogenesis | Neuroprotection |
| O60568 | PLOD3 | 4.97E+07 | Disease modifier | Neuroprotection |
| A0A0U1RQF0 | FASN | 9.66E+07 | Retinal protection | Neuroprotection |
| P14543 | NID1 | 9.43E+07 | Basal membrane | Neuroprotection |
| P06733 | ENO1 | 6.72E+08 | Hypoxia tolerance | Neuroprotection |
| P63313 | TMSB10 | 1.86E+08 | Organization of | Neuroprotection |
| | | | skeleton | |
| Q14112 | NID2 | 2.74E+09 | Basal membrane | Neuroprotection |
| A0A087X1J7 | GPX3 | 2.88E+08 | Oxidative stress | Neuroprotection |
| Q02809 | PLOD1 | 1.88E+09 | Disease modifier | Neuroprotection |
| P10909 | CLU | 4.94E+09 | Reduces immune | Neuroprotection |
| P08758 | ANXA5 | 3.07E+09 | Anticoagulant | Neuroprotection |
| Q08431 | MFGE8 | 1.73E+09 | VEGF | Neuroprotection |
| P01008 | SERPINC1 | 5.55E+09 | Anticoagulant | Neuroprotection |
| P17677 | GAP43 | 2.00E+08 | Neuron | Neuroprotection |
| | | | remodeling | |
| P06744 | GPI | 5.33E+09 | Neurotrophic | Neuroprotection |
| O75131 | CPNE3 | 1.76E+08 | RGC | Photoreceptor |
| | | | | development |
| P07195 | LDHB | 3.34E+08 | Glycolysis | Photoreceptor |
| | | | | development |
| H0YCG2 | LAMP2 | 5.87E+07 | Inner segments | Photoreceptor |
| | | | | development |
| P00338 | LDHA | 4.38E+08 | Glycolysis | Photoreceptor |
| | | | | development |
| E7EQR4 | EZR | 4.55E+08 | Rhodopsin | Photoreceptor |
| | | | | development |
| A0A2R8Y478 | CD9 | 9.38E+08 | Cell migration | Photoreceptor |
| | | | | development |
| E9PIM6 | THYI | 1.44E+09 | RGC | Photoreceptor |
| | | | | development |
| P08670 | VIM | 1.57E+09 | Muller cells | Photoreceptor |
| | | | | development |
| E9PJK1 | CD81 | 3.36E+09 | Muller, RPE | Photoreceptor |
| | | | | development |
| P11279 | LAMP1 | 5.83E+07 | Inner segments | Photoreceptor |
| | | | | development |
| P26022 | PTX3 | 2.26E+08 | Cytokines | Pro-inflammatory |
| P14174 | MIF | 2.41E+08 | Cytokine response | Pro-inflammatory |
| C9JR96 | MME | 1.74E+08 | Diabetic | Pro-inflammatory |
| | | | retinopathy | |
| P55083 | MFAP4 | 1.75E+08 | Diabetic | Pro-inflammatory |
| | | | retinopathy | |
| P09486 | SPARC | 2.33E+08 | Ocular diseases | Pro-inflammatory |
| P61586 | RHOA | 6.93E+07 | Axon retraction | Programmed Cell |
| | | | | death |
| H7BZ94 | P4HB | 8.59E+07 | Apoptosis | Programmed Cell |
| | | | | death |
| H0Y3Z3 | P4HB | 1.10E+08 | Apoptosis | Programmed Cell |
| | | | | death |
| P35908 | KRT2 | 1.83E+08 | Cornification | Programmed Cell |
| | | | | death |
| Q15582 | TGFBI | 3.35E+08 | Immune | Programmed Cell |
| | | | | death |
| G3XAL9 | SLC12A2 | 6.64E+07 | RPE | Retinal development |
| D6RBT0 | SEC31A | 3.05E+06 | Nuclear pore | Retinal development |
| | | | complex | |
| R4GN83 | BSG | 2.31E+08 | Retinal neuron | Retinal development |
| O14672 | ADAM10 | 2.81E+08 | Early retina | Retinal development |
| E5RFU4 | DPYSL2 | 5.05E+07 | RGC | Retinal development |
| D6RFI1 | DBN1 | 5.20E+07 | Whole retina | Retinal development |
| P04899 | GNAI2 | 4.32E+08 | Muller glia | Retinal development |
| Q9UBI6 | GNG12 | 2.71E+08 | Axonal guidance | Retinal development |
| P98160 | HSPG2 | 6.62E+07 | Basement | Retinal development |
| | | | membrane | |
| P36955 | SERPINF1 | 1.25E+08 | RPE | Retinal development |
| C9JIS1 | GNB2 | 5.61E+08 | Axonal guidance | Retinal development |
| Q9HAV0 | GNB4 | 5.75E+08 | Rods | Retinal development |
| P62873 | GNB1 | 8.07E+08 | Axonal guidance | Retinal development |
| P26038 | MSN | 4.86E+08 | Structure | Retinal development |
| P61769 | B2M | 4.06E+08 | Neural retina | Retinal development |
| C9J419 | PXDN | 2.50E+08 | Embryo | Retinal development |
| Q92626 | PXDN | 4.28E+08 | Embryo | Retinal development |
| K7ES78 | CAPNS1 | 5.83E+07 | Embryonic dev | Retinal development |
| C9JWG0 | UGP2 | 1.13E+08 | Rods nuclear | Retinal development |
| Q13641 | TPBG | 1.40E+08 | Rod bipolar cells | Retinal development |
| P48061 | CXCL12 | 3.65E+08 | Cell proliferation | Retinal development |
| P13612 | ITGA4 | 6.83E+07 | Integrin | Retinal Engraftment |
| | | | fibronectin | |
| A0A494C194 | PEPD | 1.21E+08 | Collagen binding | Retinal Engraftment |
| P08648 | ITGA5 | 1.63E+08 | Integrin | Retinal Engraftment |
| | | | fibronectin | |
| P06756 | ITGAV | 3.39E+08 | Integrin laminin | Retinal Engraftment |
| P50454 | SERPINH1 | 2.92E+08 | Collagen binding | Retinal Engraftment |
| H0YDX6 | CD44 | 3.80E+08 | HA binding | Retinal Engraftment |
| Q86W61 | VCAN | 2.16E+08 | HA binding | Retinal Engraftment |
| P05556 | ITGB1 | 3.39E+08 | Integrin collagen | Retinal Engraftment |
| E9PF17 | VCAN | 1.91E+08 | HA binding | Retinal Engraftment |
| P21810 | BGN | 1.92E+08 | Chondroitin | Retinal Engraftment |
| | | | sulfate | |
| O43854 | EDIL3 | 3.81E+08 | Binding | Retinal Engraftment |
| P10809 | HSPD1 | 2.54E+07 | Protection | Retinal protection |
| Q580Q6 | EFEMP1 | 1.73E+09 | Protects from | Retinal protection |
| | | | AMD | |
| H7C597 | SND1 | 2.69E+09 | Photoreceptor | Retinal protection |
| | | | protection | |
| P10599 | TXN | 4.07E+09 | RDCVF | Retinal protection |
| F8WF32 | RPN1 | 6.16E+07 | Protects from | Retinal protection |
| | | | Stargardt's | |
| O60701 | UGDH | 9.89E+07 | Protects RGC | Retinal protection |
| A0A286YFA2 | PHGDH | 3.64E+07 | Protects from | Retinal protection |
| | | | MacTel | |
| P78539 | SRPX | 1.21E+09 | Protects from RP | Retinal protection |
| P07093 | SERPINE2 | 5.83E+07 | Angiogenesis | Retinal protection |
| P63092 | GNAS | 4.12E+08 | RGC and ON | Retinal protection |
| P07900 | HSP90AA1 | 3.71E+08 | Protection | Retinal protection |
| K7EJB9 | CALR | 1.15E+09 | Protects from | Retinal protection |
| | | | CRVO | |
| P08238 | HSP90AB1 | 4.08E+08 | Protection | Retinal protection |
| P07585 | DCN | 2.52E+08 | Retinal structure | Retinal protection |
| J3QKS4 | TSG101 | 1.44E+09 | Protects RPE | Retinal protection |
| P11142 | HSPA8 | 6.27E+08 | Protection | Retinal protection |
| Q14204 | DYNC1H1 | 5.83E+07 | Rod inner/outer | Retinal protection |
| P09211 | GSTP1 | 4.52E+08 | Protects RPE | Retinal protection |
| A0A0J9YXZ5 | IQGAP1 | 1.24E+09 | Protects from | Retinal protection |
| | | | CNV | |
| H3BLV0 | CD55 | 1.36E+09 | Protects from | Retinal protection |
| | | | AMD | |
| H3BRM6 | GSTM1 | 1.77E+09 | Protects from | Retinal protection |
| | | | AMD | |
| Q9Y696 | CLIC4 | 1.92E+09 | Protects from RD | Retinal protection |
| Q13443 | ADAM9 | 4.98E+09 | Protects from | Retinal protection |
| | | | CRD | |
| A0A2R8Y484 | CD47 | 8.20E+09 | Cone survival | Retinal protection |
| F8VQ14 | CCT2 | 8.44E+09 | Protects from | Retinal protection |
| | | | LCA | |
| Q92629 | SGCD | 5.95E+10 | Protects from | Retinal protection |
| | | | AMD | |
| E9PL09 | RPS3 | 7.64E+07 | 40S subunit | Ribosome |
| | | | | Biogenesis |
| Q5T6W2 | HNRNPK | 6.38E+07 | Ribonucleoprotein | Ribosome |
| | | | | Biogenesis |
| P25398 | RPS12 | 1.36E+08 | 40S subunit | Ribosome |
| | | | | Biogenesis |
| J3KSS0 | RPL26 | 6.23E+06 | Ribonucleoprotein | Ribosome |
| | | | | Biogenesis |
| G3V1B3 | RPL21 | 5.83E+07 | Ribonucleoprotein | Ribosome |
| | | | | Biogenesis |
| B7Z645 | SYNCRIP | 6.21E+07 | Ribonucleoprotein | Ribosome |
| | | | | Biogenesis |
| M0QZC5 | RPS11 | 1.29E+08 | 40S subunit | Ribosome |
| | | | | Biogenesis |
| H7C2W9 | RPL31 | 1.34E+08 | Ribonucleoprotein | Ribosome |
| | | | | Biogenesis |
| H0YEN5 | RPS2 | 1.43E+08 | 40S subunit | Ribosome |
| | | | | Biogenesis |
| M0QZN2 | RPS5 | 2.34E+08 | 40S subunit | Ribosome |
| | | | | Biogenesis |
| F8W7C6 | RPL10 | 7.64E+07 | Ribonucleoprotein | Ribosome |
| | | | | Biogenesis |
| J3JS69 | RPS18 | 1.29E+08 | 40S subunit | Ribosome |
| | | | | Biogenesis |
| C9J9K3 | RPSA | 1.64E+08 | 40S subunit | Ribosome |
| | | | | Biogenesis |
| P05387 | RPLP2 | 5.37E+07 | 60s subunit | Ribosome |
| | | | | Biogenesis |
| D6RBD0 | GNB2L1 | 1.61E+08 | 40S subunit | Ribosome |
| | | | | Biogenesis |
| J3KMX5 | RPS13 | 1.82E+08 | 40S subunit | Ribosome |
| | | | | Biogenesis |
| P62701 | RPS4X | 1.02E+08 | 40S subunit | Ribosome |
| | | | | Biogenesis |
| P62851 | RPS25 | 1.15E+07 | 40S subunit | Ribosome |
| | | | | Biogenesis |
| M0QX76 | RPS16 | 2.40E+08 | 40S subunit | Ribosome |
| | | | | Biogenesis |
| D6RGE0 | RPS3A | 3.2E+08 | 40S subunit | Ribosome |
| | | | | Biogenesis |
| P05386 | RPLP1 | 6.90E+08 | 60s subunit | Ribosome |
| | | | | Biogenesis |
| ESRIP1 | RPS20 | 7.53E+07 | 40S subunit | Ribosome |
| | | | | Biogenesis |
| Q5JR95 | RPS8 | 2.36E+08 | 40S subunit | Ribosome |
| | | | | Biogenesis |
| P13639 | EEF2 | 4.21E+08 | mRNA | Ribosome |
| | | | | Biogenesis |
| H7BYG8 | LTN1 | 7.39E+09 | Ribonucleoprotein | Ribosome |
| | | | | Biogenesis |
| P09936 | UCHL1 | 1.21E+08 | Stroke treatment | Tissue Regeneration |
| Q9NZM1 | MYOF | 1.03E+08 | Repair membrane | Tissue Regeneration |
| P14625 | HSP90B1 | 2.47E+08 | neural chaperon | Tissue Regeneration |
| P29966 | MARCKS | 8.18E+08 | Neurons and | Tissue Regeneration |
| | | | axons | |
| J3QSU6 | TNC | 7.23E+08 | Neuron guidance | Tissue Regeneration |
| P04406 | GAPDH | 2.29E+09 | Regenerate | Tissue Regeneration |
| Q15063 | POSTN | 1.10E+09 | Stem neuron | Tissue Regeneration |
| | | | proliferation | |
| B1ALD9 | POSTN | 1.10E+09 | Stem neuron | Tissue Regeneration |
| | | | proliferation | |
| B1AHC9 | XRCC6 | | DNA repair | Tissue Regeneration |
| P00742 | F10 | 7.20E+07 | Cell migration | Tissue Regeneration |
| C9JKI3 | CAV1 | 1.59E+08 | In the retina, | Tissue Regeneration |
| | | | homeostasis | |

### Example 3. In Vivo Evaluation of/Rod-EVs

Rho−/− mice are knockout mice that lack the gene for rhodopsin, a light-sensitive protein found in the rods of the retina. Rhodopsin is essential for vision in low light conditions. Therefore, Rho−/− mice have impaired vision in dim light, and their rods do not function properly. They are commonly used in research related to retinal degeneration. P23h homozygous mice, on the other hand, have a mutation in the rhodopsin gene that causes it to produce a defective form of rhodopsin protein. This mutation is similar to the one found in humans with autosomal dominant retinitis pigmentosa (RP). P23h homozygous mice have progressive retinal degeneration and vision loss, similar to RP patients. They are used in research related to understanding the molecular mechanisms underlying RP and for testing potential treatments. So, while both rho−/− and P23h homozygous mice are used in vision-related research, their genetic modifications and associated characteristics are different.

Due to this mutation, rod photoreceptors malfunction and start to degenerate as soon as 3 weeks post-natal. This is followed by the death of cones starting at 5-6 weeks post-natal. To neuroprotect the retina, we injected 4 week old nice with the technology cell+gel (gel composed of gelatin and hyaluronic acid combined at 5.5% w/v concentration and crosslinked using HRP and H₂O₂) to potentially halt and slow down the degeneration of these mice, with a focus on cone neuroprotection. We tested the functional and retinal behavior of these mice each week post-injection. The functional behavior testing, FIGS. 22A-B, was performed using optomotor testing to measure both visual acuity and contrast sensitivity. We confirmed, as shown in previous papers (Xiao et al., Invest Ophthalmol Vis Sci. 2019 Oct. 1; 60(13):4196-4204), that visual acuity in Rho−/− does not decrease prior to 12 weeks, independent of the injection group. However, we observed a decrease in contrast sensitivity for SHAM group as soon as 2 weeks post-injection. The cell+gel treatment was able to stop that decrease and delay it by at least 1 month as seen in the significant differences at weeks 8 and 9. While this is an ongoing study, we expect to obtain high durability in neuroprotecting the functional behavior of these mice until at least 12-14 weeks.

These results were confirmed by the cone function test performed using electroretinogram (ERG), as seen in FIGS. 23A-C. We focused on Photopic B-wave, which is representative on cone function in the retina. As seen in FIG. 23A, a significant increase in cone function was observed at 7- and 8-weeks following injection, which was also measured using the percent increase (FIG. 23B) and confirmed with the ERG traces (FIG. 23C).

Overall, this ongoing study suggests that our gel+cell can neuroprotect the function of cones in the retina of degenerative Rho−/− mice.

As stated in Methods, P23H have a genetic mutation with a misfolding of rhodopsin protein. We performed a similar study as for Rho−/−, knowing that P23H have a faster degenerative profile. While no Scotopic wave was observed in P23H, we were able to measure, using ERG, the photopic B wave of 3 different groups (SHAM, cells, cell+gel) as seen in FIGS. 24A-B. A significant improvement in Photopic B-wave was observed as soon as 1 week after injection for both cell and cell+gel compared to SHAM group.

These results show the efficacy of this treatment even at a late-stage degenerative retina.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of providing a population of progenitor rod photoreceptor cells (PRPs), the method comprising:
providing an initial population comprising retinal cells from a mammal, preferably a fetal mammal;
isolating cells expressing CD73 and CD276 from the initial population to provide a substantially purified population of PRPs; and
optionally culturing the substantially purified population.

2. The method of claim 1, wherein isolating cells expressing CD73 and CD276 comprises using fluorescence activated cell sorting (FACS) or Magnetic-activated cell sorting (MACS), optionally in a microfluidic device.

3. The method of claim 1, further comprising culturing the initial population comprising retinal cells before isolating cells expressing CD73 and CD276.

4. The method of claim 1, wherein the substantially purified population of PRPs is cultured in media comprising serum or serum replacement, EGF, and FGF.

5. The method of claim 1, wherein the substantially purified population of PRPs is cultured in hypoxic conditions.

6. The method of claim 1, wherein the initial population comprising retinal cells is from a fetal mammal.

7. The method of claim 1, wherein the mammal is a human.

8. A substantially purified population of PRPs produced by the method of claim 1, preferably wherein at least 75% of the population comprises cells expressing CD73 and CD276.

9. (canceled)

10. A method of treating a subject who has a condition associated with loss of retinal rod photoreceptors, the method comprising administering to the subject a therapeutically effective amount of PRPs obtained by the method of claim 1, preferably wherein at least 75% of the population comprises cells expressing CD73 and CD276.

11. A method of providing extracellular vesicles (EVs) from rod photoreceptor progenitor cells, the method comprising:
providing the substantially purified population of PRPs of claim 8;
maintaining the substantially purified population of PRPs in culture in media, preferably wherein the media is in contact with the PRPs in the culture for at least 12, 18, 24, 36, 48, or 72 hours;
removing the media from the culture; and
isolating EVs from the media.

12. The method of claim 11, wherein isolating EVs comprises ultracentrifuging the media to obtain a pellet comprising EVs.

13. The method of claim 11, further comprising lyophilizing the isolated EVs.

14. A composition comprising EVs obtained by the method of claim 11.

15. The composition of claim 14, wherein the EVs are in a polymeric scaffold.

16. The composition of claim 15, wherein the polymeric scaffold comprises polycaprolactone (PCL), polylactic-glycolic acid (PLGA), polyethylene glycol (PEG), polylactic acid (PLA), polyetherimide (PEI), PNIPAAM, or hydroxy ethyl-methacrylate.

17. The composition of claim 14, for use in treating a subject who has a condition associated with loss of retinal rod or cone photoreceptors.

18. A method of treating a subject who has a condition associated with loss of retinal rod or cone photoreceptors, the method comprising administering to the subject a therapeutically effective amount of PRPs obtained by the method of claim 11.

19. The method of claim 10, wherein the condition associated with loss of retinal rod or cone photoreceptors is an inherited retinal degenerative disease (IRD), optionally cone-rod dystrophy, retinitis pigmentosa, or Stargardt's disease; or macular degeneration.
